(19) United States
(12) Patent Application Publication
(10) Pub. No.: US 2007/0116669 A1
(43) Pub. Date: May 24, 2007

(54) Interferon-inducible protein-10 (IP-10 or CXCL10) chemokine analogs for the treatment of human diseases

(75) Inventors: Ahmed Merzouk, Richmond (CA); Donald Wong, Vancouver (CA); Hassan Salari, Vancouver (CA)

Correspondence Address: PERKINS COIE LLP, P.O. BOX 2168, MENLO PARK, CA, 94026, US

(73) Assignee: Chemokine Therapeutics Corporation

(21) Appl. No.: 11/590,210

(22) Filed: Oct. 30, 2006

Related U.S. Application Data
(63) Continuation-in-part of application No. 11/494,232, filed on Jul. 26, 2006.
Application No. 11/494,232 is a division of application No. 10/243,795, filed on Sep. 13, 2002, now Pat. No. 7,091,310.

(51) Int. Cl. A61K 38/19; C07K 14/52; C07K 16/24; A61K 39/395

(52) U.S. Cl. 424/85.1; 530/351; 424/145.1; 530/388.23

(57) ABSTRACT

This invention is directed to peptide analogs of interferon-inducible protein-10 (IP-10 or CXCL10) chemokine that bind to the CXCR3 receptor or any other receptor in which IP-10 analogs can bind to as a ligand, such that the analogs can be designed to serve as agonists or antagonists of IP-10 chemokine. The analogs can be used to prevent, treat, or ameliorate the symptoms of, a disease.

### CROSS-REFERENCE

This application is a continuation-in-part of co-pending U.S. patent application Ser. No. 11/494,232 filed Jul. 26, 2006, which is a divisional of U.S. patent application Ser. No. 10/243,795, filed Sep. 13, 2002, now U.S. Pat. No. 7,091,310 issued Aug. 15, 2006, all of which are hereby incorporated by reference in their entirety.

### SEQUENCE LISTING

This application contains a “lengthy” sequence listing which has been submitted as a CD-R in lieu of a printed paper copy and is hereby incorporated by reference in its entirety.

### FIELD OF THE INVENTION

This invention is directed to peptide analogs of interferon-inducible protein-10 (IP-10 or CXCL10) chemokine that bind to the CXCR3 receptor or any other receptor in which IP-10 analogs can bind to as a ligand, such that the analogs can be designed to serve as agonists or antagonists of IP-10 chemokine. The analogs can be used to prevent, treat, or ameliorate the symptoms of, a disease.

### BACKGROUND OF THE INVENTION

Chemokines (chemoattractant cytokines) are a family of homologous serum proteins of between 7 and 16 kDa, which were originally characterized by their ability to induce migration of leukocytes. Most chemokines have four characteristic cysteines (Cys), and depending on the motif displayed by the first two cysteines, they have been classified into CXC or alpha, CC or beta, C or gamma, and CX3C or delta chemokine classes. Two disulfide bonds are formed between the first and third cysteine and between the second and fourth cysteine. In general, it was thought that the disulfide bridges were required, and Clark-Lewis and co-workers reported that, at least for IP-10, the disulfide bridges are critical for chemokine activity (Clark-Lewis et al., J. Biol. Chem. 269:16075-16081, 1994). The only exception to having four cysteines is lymphotactin, which has only two cysteine residues. Thus, lymphotactin manages to retain a functional structure with only one disulfide bond.

In addition, the CXC, or alpha, subfamily has been divided into two groups depending on the presence of the ELR motif (Glu-Leu-Arg) preceding the first cysteine: the ELR-CXC chemokines and the non-ELR-CXC chemokines (see, e.g., Clark-Lewis, supra, and Belperio et al., “CXC Chemokines in Angiogenesis,” J. Leukoc. Biol. 68:1-8, 2000).

Chemokines have been shown to be useful in therapeutic applications. For example, the chemokine SDF-1 has been shown to enhance platelet production (Lane et al., Blood 96:4152-59, 2000) and B-cell production (Nagasawa, T., Int. J. Hematol. 72:408-11, 2000), inter alia. Other chemokine functions are reviewed in Schwarz and Wells (Schwarz and Wells, Nat. Rev. Drug Discov. 1:347-58, 2002). Glimm and colleagues reported for example, that SDF-1 arrests hematopoietic stem cell cycling, thus allowing a better transfection of these cells with gene constructs for the purpose of gene therapy (Glimm H. et al., “Ex vivo treatment of proliferating human cord blood stem cells with stroma-derived factor-1 enhances their ability to engraft NOD/SCID mice,” Blood 99(9):3454-57, 2002). All of the above references are incorporated by reference herein their entirety, including any drawings, tables, and figures.

Interferon-inducible protein-10 (IP-10 or CXCL10) is induced by interferon-gamma and TNF-alpha, and is produced by keratinocytes, endothelial cells, fibroblasts and monocytes. IP-10 is thought to play a role in recruiting activated T cells to sites of tissue inflammation (Dufour, et al., “IFN-gamma-inducible protein 10 (IP-10; CXCL10)-deficient mice reveal a role for IP-10 in effector T cell generation and trafficking,” J Immunol., 168:3195-204, 2002). In addition, IP-10 may play a role in hypersensitivity. It may also play a role in the genesis of inflammatory demyelinating neuropathies (Kieseier, et al., “Chemokines and chemokine receptors in inflammatory demyelinating neuropathies: a central role for IP-10, “Brain 125:823-34, 2002).

Research has shown that IP-10s may be useful in improving the engraftment of stem cells following transplantation (Nagasawa, 2000), as well as in mobilizing stem cells (Gazitt, Y., J. Hematother Stem Cell Res 10:229-36, 2001; Hattori et al., Blood 97:3354-59, 2001) and enhancing anti-tumor immunity (Nomura et al., Int. J. Cancer 91:597-606, 2001; Mach and Dranoff, Curr. Opin. Immunol. 12:571-75, 2000). The biological activity of chemokines has been discussed, for example, in reports known to those of skill in the art (Bruce, L. et al., “Radiolabeled Chemokine binding assays,” Methods in Molecular Biology (2000) vol. 138, pp129-134, Raphaele, B. et al. “Calcium Mobilization,” Methods in Molecular Biology (2000) vol. 138, pp143-148, Paul D. Ponath et al., “Transwell Chemotaxis,” Methods in Molecular Biology (2000) vol. 138, ppl 13-120 Humana Press. Totowa, N.J.).

Accordingly, one of skill will appreciate novel IP-10 analogs that bind to the CXCR3 receptor or any other receptor in which IP-10 analogs can be a ligand, such that the analogs can be designed to serve as agonists or antagonists of IP-10 chemokine.

### SUMMARY OF THE INVENTION

The present invention is directed to an IP-10 analog that can serve as an agonist or antagonist in the treatment of disease. More particularly, the invention is directed to a composition comprising an IP-10 chemokine analog having a length ranging from about 21 to about 34 amino acids and comprising a first conserved sequence consisting of the IP-10 chemokine residues 1-15 (SEQ ID NO: 1646), and conservatively modified variants thereof; a second conserved sequence consisting of the IP-10 chemokine residues 66-71 (SEQ ID NO: 1647), and conservatively modified variants thereof; and, an optional linker having up to 4 amino acids. The N-terminus of the N-terminal region can consist of a hydrogen or can be modified using an N-terminal modifier comprising a component selected from a group consisting of a poly(ethylene glycol) or derivative thereof, a glycosaminoglycan, a diagnostic label, a radioactive group, an acyl group, an acetyl group, a peptide, and a modifier capable of reducing the ability of the IP-10 analog to act as a substrate for arninopeptidases. The linker can be selected from a group consisting of (a) up to four natural amino acids, and (b) any non-natural amino acid having the following structure: 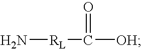
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

In some embodiments, the IP-10 analog can have a length of about 26 to 32 amino acid residues and comprise an N-terminal region consisting essentially of SEQ ID NO: 1646 and conservatively modified variants thereof in residues 1-15, and a C-terminal region comprising SEQ ID NO: 1647 and conservatively modified variants thereof. In some embodiments, the linker is 11-aminoundecanoic acid.

In many embodiments, the IP-10 analogs can be comprised of an amino acid sequence selected from a group consisting of SEQ ID NO: 1641, SEQ ID NO: 1642, SEQ ID NO: 1643, SEQ ID NO: 1644, and SEQ ID NO: 1645. In some embodiments, the IP-10 analogs can be comprised of an amino acid sequence selected from a group consisting of SEQ ID NOs:296 through 349, 404 through 457, 494 through 548, 675 through 728, variants b134 through b187, b242 through b295, b332 through b385, and b512 through b566, inclusive. These amino acid sequences can contain variables Xaa₁, Xaa₂, Xaa₃, and Xaa₄, wherein, Xaa₁, Xaa₂, Xaa₃, and Xaa₄ can each be independently selected from a group consisting of any natural amino acid or any non-natural amino acid having the following structure: 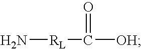
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

In many embodiments, the invention includes a method of increasing the IP-10-mediated activity of a cell having a receptor capable of binding to an IP-10 analog, comprising binding the receptor to the IP-10 analog described above. In some embodiments, the invention includes an antibody produced using the IP-10 analog described above as an antigen. In these embodiments, the antibody can be polyclonal, monoclonal, and/or humanized.

### BRIEF DESCRIPTION OF THE DRAWINGS

FIG. 1 illustrates the induction of [Ca²⁺]i mobilization by select IP-10 analogs at a concentration of 100 μM according to some embodiments of the present invention.

### DETAILED DESCRIPTION OF THE INVENTION

The present invention generally relates to the design, preparation, derivation, and use of amino acid sequences in the prevention, treatment, and ameliorization of diseases and disorders. Generally speaking, this invention is directed to the design, synthesis, and use of IP-10 analogs which bind to an IP-10 chemokine receptor, such as a CXCR3 receptor or any other receptor to which IP-10 binds, such that the IP-10 analogs can be designed to affect the activity of the receptor.

An IP-10 chemokine receptor, for example, can by any receptor recognized in the art as binding to an IP-10 chemokine, as well as any other binding moiety capable of binding to an IP-10 chemokine or IP-10 analog, wherein the activity of the receptor can be increased or decreased. In some embodiments, the terms “activity,” “activate,” “activated,” “activating,” “activation,” and the like, can refer to a cellular or extracellular function of an IP-10 chemokine receptor.

In some embodiments, biological activity can include, for example, receptor binding, chemotaxis, and calcium mobilization, as well as other activities known to those of skill in the art that are affected by the presence of a binding peptide. The IP-10 chemokine receptor function can include catalytic activity from the interaction with a natural binding partner. In some embodiments, an IP-10 mimetic activates the catalytic activity of a chemokine receptor. In some embodiments, an IP-10 mimetic inhibits the catalytic activity of a chemokine receptor. In some embodiments, the activation or inhibition of a chemokine receptor can be dependent on the concentration of an IP-10 mimetic.

A recognized sequence of the human IP-10 chemokine is as follows:

(SEQ ID NO:2)
Val-Pro-Leu-Ser-Arg-Thr-Val-Arg-Cys-Thr-Cys-Ile-Ser-Ile-Ser-Asn-
Gln-Pro-Val-Asn-Pro-Pro-Arg-Ser-Leu-Glu-Lys-Leu-Glu-Ile-Ile-
Pro-Ala-Ser-Gln-Phe-Cys-Pro-Arg-Val-Glu-Ile-Ile-Ala-Thr-Met-
Lys-Lys-Lys-Gly-Glu-Lys-Arg-Cys-Leu-Asn-Pro-Glu-Ser-Lys-Ala-
Ile-Lys-Asn-Leu-Leu-Lys-Ala-Val-Ser-Lys-Glu-Met-Ser-Lys-Arg-
Ser-Pro

The products of the present invention can be referred to by various terms including, but not limited to, “analogs,” “mimetics, “peptidomimetics,” “peptides,” “polypeptides,” “designer peptides,” “IP-10 chemokine mimetics,” “IP-10 mimetics,” “IP-10 chemokine analogs,” “IP-10 analogs,” and “IP-10 chemokine derivatives.” These terms can be used interchangeably in many embodiments and, in some embodiments, denote equivalent compounds. The IP-10 analogs can include, but are not limited to, the sequences described herein, variants and conservately modified variants thereof, and can also include additional elements such as R-group substituents and linkers, examples of which are taught in the present application.

The term “variant” refers to modifications to a peptide that allows the peptide to retain its binding properties, and such modifications include, but are not limited to, conservative substitutions in which one or more amino acids are substituted for other amino acids; deletion or addition of amino acids that have minimal influence on the binding properties or secondary structure; conjugation of a linker; post-translational modifications such as, for example, the addition of functional groups. Examples of such post-translational modifications can include, but are not limited to, the addition of modifying groups described below through processes such as, for example, glycosylation, acetylation, phosphorylation, modifications with fatty acids, formation of disulfide bonds within and between peptides, biotinylation, PEGylation, and combinations thereof

The term “conservatively modified variant” refers to a conservative amino acid substitution, which is an amino acid substituted by an amino acid of similar charge density, hydrophilicity/hydrophobicity, size, and/or configuration such as, for example, substituting valine for isoleucine. In comparison, a “non-conservatively modified variant” can refer to a non-conservative amino acid substitution, which can include, in some embodiments, an amino acid substituted by an amino acid of differing charge density, hydrophilicity/hydrophobicity, size, and/or configuration such as, for example, substituting valine for phenyalanine. One of skill will recognize that there is more than one convention for identifying conservatively modified variants.

The IP-10 chemokine and its analogs can affect biological functions and can be designed to include a wide variety of modifications to provide, for example, a diagnostic, therapeutic and/or prophylactic activity in a subject. The term “subject” and “patient” can be used interchangeably in the present invention and, in most embodiments, refer to an animal such as a mammal including, but not limited to, non-primates such as, for example, a cow, pig, horse, cat, dog, rat and mouse; and primates such as, for example, a monkey or a human.

The IP-10 analogs can act as agonists or antagonists for an IP-10 chemokine in many embodiments. In some embodiments, the N-terminal region, the C-terminal region, or both, of an IP-10 chemokine can bind to an IP-10 receptor such as, for example, a CXCR3 receptor. In some embodiments, the beta sheet structure that connects the two termini may play a role in the stabilization of the IP-10 receptor and can help assure that the termini are in the proper conformation.

Examples of these analogs include compounds that contain structures corresponding to various select regions and combinations of regions within the IP-10 chemokine. In some embodiments, the IP-10 analog comprises an N-terminal region joined with a C-terminal region, wherein the means for joining the two regions is a linker. In some embodiments, the amino acid residues of the IP-10 analog can be cyclized, e.g., by etherification of lysine and serine residues or by other means described infra or known in the art.

In some embodiments, the IP-10 analog comprises a sequence derived from a wild-type IP-10 chemokine sequence but with one or more of the cysteines replaced with another amino acid, which can be a natural or non-natural amino acid. Some embodiments include IP-10 analogs comprising an N-terminal region, an internal region containing up to three anti-parallel β-sheets, a C-terminal region containing an α-helical structure, a combination of the N- and C-terminal regions linked together directly, a combination of an N-terminal and an internal region, a combination of a C-terminal region and an internal region, a combination of an N-terminal, internal and C-terminal region, and any combination thereof.

In some embodiments, the N-terminal, internal and C-terminal regions of the IP-10 analogs may be 2, 3, 4, 5, 6, 7, 8, 9, 10, 11, 12, 13, 14, 15, 16, 17, 20, 25, 30, 35, 40, 41, or 45 amino acids in length. In some embodiments, the IP-10 analogs can be 10-15, 12-17, 15-20, 20-25, 25-30, 30-35, 35-40, 30-50, 30-70, or any range therein, amino acids in length.

Methods of Use

The IP-10 analogs can be used in treating, preventing, or ameliorating the symptoms of, several diseases. The terms “disease,” “disorder,” and “abnormal condition” can be used interchangeably in many embodiments and generally refer to a deviation from a normal function in an organism. An abnormal condition can comprise deviations, for example, in cell proliferation, cell differentiation, cell survival, cell migration or movement, or the activities of enzymes within a cell.

In some embodiments, the diseases and disorders may include, but are not limited to, hyperproliferative disorders, such as inflammatory conditions, autoimmune disorders, cancers, and vascular conditions; and neuropathies, such as inflammatory demyelinating neuropathies. In some embodiments, the diseases or disorders can include, but are not limited to, cardiovascular disease, neurological disorders, infectious disease, and inflammatory disorders that include rheumatoid arthritis, chronic inflammatory bowel disease, chronic inflammatory pelvic disease, multiple sclerosis, asthma, osteoarthritis, atherosclerosis, psoriasis, rhinitis, autoimmunity, organ transplant rejection, and genetic diseases. In some embodiments, the compositions can be used to increase the hemocrit, assist in mobilizing and recovering stem cells or progenitor cells, stimulate the production of blood cells, assist in vaccine production, assist in gene therapy, or increase apoptosis in tumors, or otherwise stimulate the immune system to effectuate tumor destruction.

The therapeutic and prophylactic effects of IP-10 mimetics can include, but are not limited to, one or more of the following: (a) an increase or decrease in the number of cells present at a specified location; (b) an increase or decrease in the ability of cells to migrate; (c) an increase or decrease in the response of cells to a stimulus; (d) an increase or decrease in the proliferation, growth, and/or differentiation of cells; (e) an inhibition or acceleration of apoptosis; (f) an ameliorization of one or more symptoms of disease; (g) an enhancement or inhibition of a cell function; and (h) an activation or inhibition of enzyme activity in cells. An increase in the number of cells is “cell expansion,” and the IP-10 mimetics can cause this increase to occur in vivo or ex vivo. Accordingly, the IP-10 mimetics can be used to create mammal cells for diagnostic, prophylactic, therapeutic applications as well as commercial applications that include, but are not limited to, medicinal research and development of products such as, for example, vaccines.

Generally speaking, a function or activity of a chemokine receptor can be increased or decreased by contacting the receptor with an IP-10 mimetic to increase or decrease the probability of forming a complex between the receptor and a natural binding partner. The term “natural binding partner” includes, but is not limited to, G proteins, polypeptides, lipids, small molecules, or nucleic acids that bind to chemokine receptors in cells or in the extracellular environment. The term natural binding partner also includes substrates that can be acted upon by a chemokine receptor. The terms “modulate,” “modulating,” and the like, can be used in some embodiments to refer to altering the function or activity of a chemokine receptor by contacting it with a chemokine or chemokine analog and thus increasing or decreasing the probability that a complex forms between the receptor and a natural binding partner. The term “complex” refers to an assembly of at least two molecules bound to one another. The concentration of the IP-10 analog can be adjusted to control the degree of the effect.

The term “natural binding partner” refers to G proteins, polypeptides, lipids, small molecules, or nucleic acids that bind to IP-10 chemokine receptors in cells or in the extracellular environment. The term natural binding partner includes a substrate to be acted upon by the IP-10 chemokine receptor. A change in the interaction between an IP-10 chemokine receptor and a natural binding partner can manifest itself as an increased or decreased probability that the interaction forms, or an increased or decreased concentration of IP-10 chemokine receptor/natural binding partner complex. This can result in a decreased or increased activity of the IP-10 chemokine receptor.

In some embodiments, the terms “contact,” “contacting,” and the like, can refer to binding an IP-10 analog to a cell, and this can occur in vivo or in vitro, for example. In these embodiments, for example, an IP-10 analog can be contacted with a receptor by adding together a solution or a composition comprising the IP-10 chemokine or IP-10 analog with a liquid medium bath containing a polypeptide or a cell comprising an IP-10 chemokine receptor. The solution comprising the IP-10 chemokine or IP-10 analog may also comprise another component, such as dimethyl sulfoxide (DMSO), which facilitates the uptake of the IP-10 chemokine or IP-10 analog into the cells of the methods. The solution comprising the IP-10 chemokine or IP-10 analog may be added to the medium bathing the cells by utilizing a delivery apparatus, such as a pipette-based device or syringe-based device.

In some embodiments, an IP-10 mimetic can increase the probability of forming a complex between a chemokine receptor and a natural binding partner. In some embodiments, an IP-10 mimetic can decrease the probability of forming a complex between a chemokine receptor and a natural binding partner. In some embodiments, the concentration of an IP-10 mimetic can control the probability that a complex between a chemokine receptor and a natural binding partner will form. The term “complex” refers to a binding between at least two molecules.

In some embodiments, the IP-10 analogs can affect the formation of signal transduction complexes. A signal transduction complex often contains at least two protein molecules bound to one another. For instance, a protein tyrosine receptor protein kinase, GRB2, Sos (son of sevenless protein), Raf, and Ras assemble to form a signal transduction complex in response to a mitogenic ligand. In some embodiments, for example, a G protein can be bound to an IP-10 chemokine receptor, and the IP-10 analog can affect the formation of the complex.

The IP-10 mimetics of the present invention can be administered to treat, prevent, or ameliorate the symptoms of neurological disorders. Due to a traditional view that cells cannot be replaced, current treatments have focused on preventing cell death, but evidence has shown that some cells have the ability to regenerate. Recent research in the use of human stem cells and progenitor cells has catapulted cell replacement therapy to the position of a real alternative to current treatments such as, for example, treatments for neurodegenerative and other diseases. The term “stem cell” can refer to a cell that can be self-renewing over a prolonged time period and can generate multiple phenotypes in response to an exogenous signal. The term “progenitor cell” can refer to a cell that is more restricted in its differentiation capability and undergoes only limited self-renewal. For example, an eosinophil progenitor must become an eosinophil—it can't become a neutrophil, although both are granulocytes.

Due to the belief that brain and spinal cord cells cannot be replaced, treatments of neurological disorders have, as described above, focused on preventing neuronal death. Evidence has proven that some cells of the central nervous system such as, for example, neuronal stem cells, can regenerate. The term “neuronal stem cell” can refer to a cell that can self-renew, give rise to other cells through asymmetric cell division/differentiation, generate neural tissue, or are derived from the nervous system. Neuronal stem cells exist in a post-implantation developing mammalian nervous system as fetal stem cells and in the adult nervous system as adult stem cells. Neurones and neuronal progenitors can also be derived from the more primitive embryonic stem cell of the inner cell mass (ICM) of the pre- or peri-implantation embryo. For example, progenitor cells are found in an adult mammalian spinal cord and can be manipulated to mature into all of the major cell types that are found in the brain and spinal cord such as, for example, neurons and glial cells. These progenitor cells can be manipulated within the brain and spinal cord, for example, by administering growth factors.

The term “embryonic stem cell” refers to a primitive form of cell that can be isolated from an embryo days after conception, is unformed and unprogrammed, and has an innate ability to develop into any other type of cell in the body such as, for example, new brain cells, insulin-producing pancreas cells, heart muscle, and other tissues that could take the place of damaged or diseased cells. Accordingly, the implanting of embryonic stem cells can create not only neurons but also cells, tissues and organs of all other systems. When implanted into degenerating areas of the brain, for example, these cells have the capacity to acquire relevant cellular phenotype of the degenerated region and take on functions of cells that were lost through disease.

Examples of neurological disorders include, but are not limited to, Parkinson's disease, Alzheimer's disease, multiple sclerosis, and any other conditions associated with neuronal stem cells. Parkinson's disease, for example, can be treated with limited success in some cases by drugs including, but not limited to, L-beta-3,4-dihydroxyphenylalanine hydrochloride (L-Dopa). Alzheimer's disease, for example, is irreversible despite treatments with drugs including, but not limited to, ARICEPT®. Such treatments have had limited success in ameliorating symptoms and slowing progression of the disease, but most patients with Alzheimer's disease require some sort of palliative care. Multiple sclerosis, for example, is sometimes responsive to the anti-inflammatory drugs including, but not limited to, interferon-β, but patients with multiple sclerosis remain incurable over the long term. As a result, subjects with multiple sclerosis develop permanent motor, sensory and cognitive deficits. In general, the treatment and reversal of neurodegenerative diseases of the brain and spinal cord remains a formidable challenge.

In some embodiments, a treatment of a neurological disorder can include administration of a composition comprising an IP-10 mimetic. In some embodiments, a treatment of a neurological disorder can include administering a composition comprising an IP-10 mimetic before, during or after administering one more other treatments or agents. In one example, an IP-10 mimetic can be administered as an agonist to mobilize stem cells in the treatment of a disease. In these embodiments, the IP-10 mimetic can contain the N-terminal portion of the IP-10 and the C-terminal portion of the IP-10 connected by a linker.

The IP-10 mimetics of the present invention can be administered to treat, prevent, or ameliorate the symptoms of autoimmune disorders. Autoimmune disorders can be organ-specific or systemic and are provoked by different pathogenic mechanisms. Examples of organ-specific autoimmune disorders include, but are not limited to, diabetes, hyperthyroidism, autoimmune adrenal insufficiency, pure red cell anemia, multiple sclerosis, and rheumatic carditis. Examples of systemic autoimmune diseases include systemic lupus erythematosus, rheumatoid arthritis, chronic inflammation, Sjogren's syndrome polymyositis, dermatomyositis and scleroderma. In some embodiments, a treatment of an autoimmune disorder can include administering a composition comprising an IP-10 mimetic. In some embodiments, a treatment of an autoimmune disorder can include administering a composition comprising an IP-10 mimetic before, during or after administering one or more other agents that are diagnostic, therapeutic, or a combination thereof.

The IP-10 mimetics of the present invention can be administered to treat, prevent, or ameliorate the symptoms of inflammatory disorders. Inflammatory disorders are manifested by an inflammatory response, which is initiated by injury that can be a result of trauma, ischemia or the introduction of foreign particles; and infection, which can be bacterial or viral. Inflammation includes a complex series of events including chemical mediators such as cytokines and prostaglandins; and inflammatory cells such as, for example, leukocytes.

The inflammatory response is a delicate interplay between the humoral and cellular immune elements that enables elimination of harmful agents and initiation of tissue repair. Unfortunately, the inflammatory response can be a disorder in its own right by causing considerable, and potentially more, damage to tissue than the disorder that initiated the inflammatory response. Examples of inflammatory disorders include, but are not limited to, acute and chronic inflammatory diseases such as, for example, arthritis, atheromas, colitis, chronic inflammatory bowel disease, chronic inflammatory pelvic disease, asthma, psoriasis, and rhinitis. Current treatments for inflammatory disorders include the use of non-steroidal anti-inflammatory drugs, which can cause, inter alia, gastrointestinal side effects; corticosteroids, which can cause, inter alia, an increased risk of infection; and immunosuppressive agents, which can leave a subject defenseless to infections.

In some embodiments, a treatment of an inflammatory disorder can include administering a composition comprising an IP-10 mimetic. In some embodiments, a treatment of an inflammatory disorder can include administering a composition comprising an IP-10 mimetic, either an agonist or antagonist, before, during or after administering one or more other agents. In some embodiments, the IP-10 mimetic can contain the N-terminal portion of the IP-10 and the C-terminal portion of the IP-10 connected by a linker.

The IP-10 mimetics of the present invention can be administered to transplant recipients. Transplant rejections occur in recipients receiving tissue from donors that differ genetically, and such rejections are mediated by T-cell dependent mechanisms. Immunosuppressive agents such as calcineurin phosphatase inhibitors and glucocorticosteroids are administered to transplant recipients to prevent allograft rejection. Immunosuppressive agents have a short lasting effect, so transplant recipients normally require life-long treatment with such agents. Life-long treatment with immunosuppressive agents creates serious adverse effects in a recipient such as, for example, the development of infections and tumors.

In some embodiments, the IP-10 analogs can be used in the treatment of graft rejection. The IP-10 mimetics of the present invention can be administered to transplant recipients to modulate cellular response and achieve effects that are diagnostic, therapeutic, prophylactic, ameliorative or a combination thereof In some embodiments, a treatment of a transplant rejection can include administering a composition comprising an IP-10 mimetic for stem cell mobilization for transplantations. In some embodiments, a treatment of a transplant rejection can include administering a composition comprising an IP-10 mimetic before, during or after administering one or more other agents. In these embodiments, the IP-10 mimetic can contain the N-terminal portion of the IP-10 and the C-terminal portion of the IP-10 connected by a linker.

The IP-10 mimetics of the present invention can be administered to treat, prevent, or ameliorate the symptoms of cardiovascular disease. Cardiovascular disease is a broad, all-encompassing term that refers to a collection of diseases and conditions and is any disorder of the heart and blood vessels. Examples of cardiovascular disorders and treatments include, but are not limited to, any disease involving heart or vascular tissue such as, for example, atherosclerosis, and ischemic heart or vascular tissue requiring reconstruction and/or management. Traditional therapies used to treat cardiovascular disease have used either angioplasty procedures to compress blockages in arteries or coronary artery bypass grafts to provide an alternate path for blood flow around clogged vascular passageways. A latest generation of treatments includes “therapeutic angiogenesis,” which is an inducement of angiogenesis to produce new blood vessels that supplement or replace a diseased vascular passageway.

In some embodiments, the IP-10 analogs can be used to treat or manage a wide variety of vascular conditions that include, for example, atherosclerosis, restenosis, vascular disorders associated with autoimmune diseases such as systemic lupus erythematosis, and ischemia-reperfusion. In some embodiments, the IP-10 analogs can be useful in either the promotion or inhibition of angiogenesis.

In some embodiments, a treatment of a cardiovascular disorder can include administering a composition comprising an IP-10 mimetic to modulate angiogenesis and assist in the reconstruction of heart or vascular tissue, which can include the proliferation and/or mobilization of endothelial cells such as, for example, vascular endothelial cells. In some embodiments, a treatment of a cardiovascular disorder can include administering compositions comprising an IP-10 mimetic before, during or after administering one more other agents. In these embodiments, the IP-10 mimetic can contain the N-terminal portion of the IP-10 and the C-terminal portion of the IP-10 connected by a linker.

The IP-10 mimetics can be used to treat conditions associated with cancer. The term “cancer” can refer to all types of cancer or neoplasm or malignant tumors found in mammals, especially humans, and include, but are not limited to, sarcomas, leukemias, carcinomas and melanoma. The methods of the present invention include treatments that slow the growth of tumors, prevent tumor growth, induce partial regression of tumors, and induce complete regression of tumors, to the point of complete disappearance. The methods also include preventing the outgrowth of metastases derived from solid tumors.

The term “leukemia,” for example, refers broadly to progressive, malignant diseases of the blood-forming organs and is generally characterized by a distorted proliferation and development of leukocytes and their precursors in the blood and bone marrow. The major types of leukemias include acute myeloid leukemia, acute lymphoid leukemia, chronic myeloid leukemia, and chronic lymphoid leukemia. Acute leukemias are those that, if left untreated, can cause fatal complications in a very short period of time. Chronic leukemias, on the other hand, may not cause any problems at all for years. Hairy cell leukemia, a disease due to overproduction of mature lymphoid cells, affects blood, bone marrow, lymph glands, spleen and liver and is more commonly seen after age of 50.

The term “sarcoma” generally refers to a tumor which is made up of a substance like the embryonic connective tissue and is generally composed of closely packed cells embedded in a fibrillar or homogeneous substance. The term “melanoma” is taken to mean a tumor arising from the melanocytic system of the skin and other organs. The term “carcinoma” refers to a malignant new growth made up of epithelial cells tending to infiltrate the surrounding tissues and give rise to metastases.

The IP-10 analogs can be used to treat or manage a wide variety of cancers that include, but are not limited to, any of a variety of malignancies and their potential metastases, particularly solid and liquid human malignancies; and, relapses of the same. In some embodiments, the cell proliferation disorders include, but are not limited to, lung large cell carcinoma, colon adenocarcinoma, skin cancer (basal cell carcinoma and malignant melanoma), renal adenocarcinoma, promyelocytic leukemia, T cell lymphoma, cutaneous T cell lymphoma, breast adenocarcinoma, steroid sensitive tumors, hairy cell leukemia, Kaposi's Sarcoma, chronic myelogenous leukemia, multiple myeloma, superficial bladder cancer, ovarian cancer, and glioma.

In some embodiments, the cell proliferation disorders include cells derived from reproductive tissue (such as Sertoli cells, germ cells, developing or more mature spermatogonia, spermatids or spermatocytes and nurse cells, germ cells and other cells of the ovary), the lymphoid or immune systems (such as Hodgkin's disease and non-Hodgkin's lymphomas), the hematopoietic system, and epithelium (such as skin, including malignant melanoma, and gastrointestinal tract), solid organs, the nervous system, e.g. glioma (see Y. X. Zhou et al., 2002), and musculo-skeletal tissue.

In some embodiments, the cell proliferation disorders include solid tumor types, including, but not limited to, brain, including medulloblastoma, head and neck, breast, colon, small cell lung, large cell lung, thyroid, testicle, bladder, prostate, liver, kidney, pancreatic, esophogeal, stomach, ovarian, pr cervical tumors. Treatment of breast, colon, lung, and prostate tumors is particularly contemplated.

The IP-10 mimetics can be used to prevent, treat, or ameliorate the symptoms of, a variety of haematological disorders. Examples of hematological disorders include, but are not limited to, bone marrow depression, aplastic anemia, agranulocytosis, leukopenia, pancytopenia, thrombocytopenia, macrocytic or megaloblastic anemia. In some embodiments, a treatment of a hematological disorder can include managing white blood cells, platelets, red blood cells, stem cells and various progenitor subsets by administering a composition comprising an IP-10 mimetic. In some embodiments, an IP-10 mimetic can be administered as an agonist to mobilize stem cells in the treatment of a disease. In one example, an IP-10 can be administered to increase the proliferation of blood cells and/or mobilize the blood cells into the bloodstream. In some embodiments, a treatment of a hematological disorder can include administering a composition comprising an IP-10 mimetic before, during or after administration of one or more other treatments or agents. In these embodiments, the IP-10 mimetic can contain the N-terminal portion of the IP-10 and the C-terminal portion of the IP-10 connected by a linker.

The IP-10 analogs can be used to treat or manage a wide variety of conditions associated with cancers that include, but are not limited to, a diverse group of hematopoietic stem cell disorders known as myelodysplastic syndrome (MDS). Such disorders are characterized by a cellular marrow with an impaired morphology and maturation (dysmyelopoiesis), peripheral blood cytopenias, and a variable risk of progression to acute leukemia that results from an ineffective blood cell production. See The Merck Manual 953, (17^{th} ed. 1999).

Disorders associated with cancers can result from an initial hematopoietic stem cell injury that can be from a variety of causes including, but not limited to, cytotoxic chemotherapy, radiation therapy, virus, chemical exposure and genetic predisposition. A clonal mutation predominates over bone marrow and suppresses healthy stem cells. In the early stages of MDS, for example, a main cause of cytopenia is an increase in programmed cell death, or apoptosis. As the disease progresses and converts into leukemia, gene mutation rarely occurs and a proliferation of leukemic cells overwhelms the healthy marrow. The course of the disease can vary, and some cases can behave as an indolent disease, whereas others can behave aggressively with a short clinical progression into an acute form of leukemia. Subjects that survive a malignancy treatment with chemotherapy drugs such as, for example, alkylating agents, with or without radiotherapy, can have a high incidence of developing MDS or secondary acute leukemia. Examples of treatments for MDS include, but are not limited to, bone marrow transplantation, transfusions, and administration of hematopoietic growth factors and cytokines. Since the IP-10 mimetics are chemokines, they are a subspecies of cytokines.

In some embodiments, a treatment of a condition associated with cancer can include the administrating a composition comprising an IP-10 mimetic before, during or after chemotherapy or radiotherapy. In some embodiments, a treatment of a condition associated with cancer can include administering a composition comprising an IP-10 mimetic before, during or after administering one more other treatments or agents. In one example, an IP-10 mimetic can be administered as an antagonist to inhibit or prevent the proliferation of cancer cells. In another example, an IP-10 mimetic can be administered during recovery from chemotherapy and/or radiotherapy to expedite recovery of the blood count. In these embodiments, the IP-10 mimetic can contain the N-terminal portion of the IP-10 and the C-terminal portion of the IP-10 connected by a linker.

In some embodiments, the IP-10 analogs can be administered to prevent, treat, or ameliorate the symptoms of a bacterial infection, viral infection, and sepsis. The IP-10 analogs can also be instrumental in controlling tumirogenesis. Moreover, IP-10 chemokines have also been identified as playing a role in osteoporosis and, as such, the IP-10 analogs can be instrumental in the treatment of osteoporosis.

In some embodiments, the IP-10 analogs can be used in gene therapy to treat genetic disease. For example, the IP-10 analogs may prove useful in arresting the cell cycle for conducting gene therapy.

In some embodiments, the IP-10 analogs can be used to prepare vaccines, to enhance humoral antibody production, to increase antigen-presenting T-cells, to increase dendritic cells and immunological features known as vaccine induction, and combinations thereof The term “antibody” can refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art as are techniques for preparing and characterizing antibodies. In some embodiments, the IP-10 analogs taught herein can be used as antigens to produce antibodies using methods well-known to those skilled in the art. In these embodiments, the antibody can be polyclonal or monoclonal. In some embodiments, the antibody is humanized.

Synthesis and Design of the IP-10 Analogs

The amino acids used in the present invention may comprise an amino group and a carboxyl group, and the amino group may be primary or secondary. Examples of amino acids include, but are not limited to, glycine, alanine, valine, leucine, isoleucine, methionine, phenylalanine, tyrosine, aspartic acid, glutamic acid, lysine, arginine, serine, threonine, cysteine, asparagine, proline, tryptophan, histidine and combinations thereof.

In some embodiments, the amino acids may be limited to bifunctional amino acids, trifunctional amino acids, diamines, triamines, monocarboxylics, or dicarboxylics. In some embodiments, an amino acids may be limited to containing aliphatics or aromatics. In some embodiments, the amino acids may not include lysine. It is to be appreciated that one skilled in the art will recognize that some of the groups, subgroups, and individual amino acids may not be used in some embodiments of the present invention.

The natural amino acids are identified in the present application by the conventional one-letter and three-letter abbreviations as indicated below, and are preceded by “L-” to indicate their L-form and by “D-” to refer to their D form. These abbreviations are generally accepted in the peptide art as recommended by the IUPAC-IUB commission in biochemical nomenclature:

| | | | | | |
|---|---|---|---|---|---|
| Alanine | A | Ala | Leucine | L | Leu |
| Arginine | R | Arg | Lysine | K | Lys |
| Asparagine | N | Asn | Methionine | M | Met |
| Aspartic acid | D | Asp | Phenylalanine | F | Phe |
| Cysteine | C | Cys | Proline | P | Pro |
| Glutamic acid | E | Glu | Serine | S | Ser |
| Glutamine | Q | Gln | Threonine | T | Thr |
| Glycine | G | Gly | Tryptophan | W | Trp |
| Histidine | H | His | Tyrosine | Y | Tyr |
| Isoleucine | I | Ile | Valine | V | Val |
| Ornithine | O | Orn | | | |

All of the peptide sequences set out herein are written according to the generally accepted convention, whereby the N-terminal amino acid is on the left of a described sequence and the C-terminal amino acid is on the right of the described sequence.

Synthesis of the IP-10 Analogs

The IP-10 analog compounds may be prepared by standard techniques known in the art. A peptide or polypeptide component of an IP-10 analog may comprise, at least in part, a peptide synthesized using standard techniques (such as those described by Clark-Lewis, I., Dewald, B., Loetscher, M., Moser, B., and Baggiolini, M., (1994) J. Biol. Chem., 269, 16075-16081, Merrifield R. B. (1963) J Am Chem Soc., 85, 2149-2154). Automated peptide synthesizers are commercially available (e.g., Advanced ChemTech Model 396; Milligen/Biosearch 9600, Appliedbiosystems/Pioneer). The peptides and polypeptides may be assayed for IP-10 chemokine receptor agonist or antagonist activity in accordance with standard methods.

Peptides and polypeptides may be purified by HPLC and analyzed by mass spectrometry. Peptides and polypeptides may be dimerized. In one embodiment, peptides and polypeptides are dimerized via a disulfide bridge formed by gentle oxidation of the cysteines using 10% DMSO in water. Following HPLC purification, dimer formation may be verified, by mass spectrometry. One or more modifying groups may be attached to an IP-10 derived peptidic component by standard methods, for example, using methods for reaction through an amino group (e.g., the alpha-amino group at the amino-terminus of a peptide), a carboxyl group (e.g., at the carboxy terminus of a peptide), a hydroxyl group (e.g., on a tyrosine, serine or threonine residue) or other suitable reactive group on an amino acid side chain.

In some embodiments, the IP-10 analogs comprising the C-terminal and N-terminal regions joined by a linker could be cyclized. In these embodiments, the cyclization can occur in the C-terminal region using side-chain to side-chain; side-chain to scaffold or, scaffold to scaffold cyclization. In some embodiments, lactamization, etherification, or RCM (Ring Closing Methatesis) can be used to carry out this reaction.

For instance, IP-10 analogs may be cyclized using a lactam formation procedure by joining the γ-carboxy side chain or the α-carboxy moiety of glutamate (Glu) residue to the ε-amino side chain of lysine (Lys) residue, as indicated in the following sequences by underlining of linked residues. Lactams may for example be formed between glutamic acid and lysine (Lys) in the C-terminal portion of the polypeptide.

As described above, the IP-10 analogs can include conservative amino acid substitutions. In some embodiments, a conservatively modified sequence has one or more of the amino acid residues replaced by an amino acid residue having a side chain with similar properties. Families of amino acid residues having side chains with similar properties are well known in the art and there are a few different conventions for comparing amino acids. Generally speaking, these conventions typically include a characterization of the amino acids using some form of grouping by chemical structure. (Note that the numbering schemes used for the amino acids can also vary and do not always correspond to the native sequence; most often the numbering corresponds to the native sequence or to that of the analog).

In some embodiments, the amino acids can be grouped as follows: amino acids with acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in some embodiments, an amino acid residue in a chemokine can be replaced with another amino acid residue from the same side chain family. In some embodiments, the conservative substitutions include, but are not limited to, substituting a Lys with Orn or any other (L or D) natural or (L or D) non-natural amino acid having an amino group on its side chain, or a Glu with Asp. In some embodiments, the conservative substitutions include, but are not limited to, the following: Gly [symbol] , Val [symbol] Ile [symbol] Leu, Asp [symbol] Glu, Lys [symbol] Arg, Asn [symbol] Gln, and Phe [symbol] Trp [symbol] Tyr.

Recombinant Synthesis

IP-10 chemokine, IP-10 chemokine fragments, or IP-10 analogs may also be synthesized, in whole or in part, by recombinant methods using expression vectors encoding all or part of a IP-10 chemokine.

Vectors, or preferably expression vectors, may contain a gene encoding a polypeptide of the invention, a functional derivative thereof, or another useful polypeptide. These vectors may be employed to express the encoded polypeptide in either prokaryotic or eukaryotic cells.

The term “vector” in this application refers to a DNA molecule into which another DNA of interest can be inserted by incorporation into the DNA of the vector. One skilled in the art is familiar with the term. Examples of classes of vectors can be plasmids, cosmids, viruses, and bacteriophage. Typically, vectors are designed to accept a wide variety of inserted DNA molecules and then used to transfer or transmit the DNA of interest into a host cell (e.g., bacterium, yeast, higher eukaryotic cell). A vector may be chosen based on the size of the DNA molecule to be inserted, as well as based on the intended use. For transcription into RNA or transcription followed by translation to produce an encoded polypeptide, an expression vector would be chosen. For the preservation or identification of a specific DNA sequence (e.g., one DNA sequence in a cDNA library) or for producing a large number of copies of the specific DNA sequence, a cloning vector would be chosen. If the vector is a virus or bacteriophage, the term vector may include the viral/bacteriophage coat.

Following entry into a cell, all or part of the vector DNA, including the insert DNA, may be incorporated into the host cell chromosome, or the vector may be maintained extrachromosomally. Those vectors that are maintained extrachromosomally are frequently capable of autonomous replication in a host cell into which they are introduced (e.g., many plasmids having a bacterial origin of replication). Other vectors are integrated into the genome of a host cell upon introduction into the host cell, and thereby are replicated along with the host genome.

The term “expression vector” refers to a DNA construct which allows one to place a gene encoding a gene product of interest, usually a protein, into a specific location in a vector from which the selected gene product can be expressed by the machinery of the host cell, or alternately, by in vitro expression system. This type of vector is frequently a plasmid, but other forms of expression vectors, such as bacteriophage vectors and viral vectors (e.g., adenoviruses, replication defective retroviruses, and adeno-associated viruses), may be employed. The selection of expression vectors, control sequences, transformation methods, and the like, are dependent on the type of host cell used to express the gene.

### Prokaryotic Hosts

Prokaryotic hosts are, in generally, very efficient and convenient for the production of recombinant polypeptides and are, therefore, one type of preferred expression system. Prokaryotes most frequently are represented by various strains of E. coli, but other microbial strains may be used, including other bacterial strains. Recognized prokaryotic hosts include bacteria such as E. coli, Bacillus, Streptomyces, Pseudomonas, Salmonella, Serratia, and the like. However, under such conditions, recombinantly-produced polypeptides will not be glycosylated.

In prokaryotic systems, vectors that contain replication sites and control sequences derived from a species compatible with the host may be used. Preferred prokaryotic vectors include plasmids such as those capable of replication in E. coli (such as, for example, pBR322, ColEl, pSC101, pACYC 184, pVX, pUC118, pUC119 and the like). Suitable phage or bacteriophage vectors may include λgt10, λgt11, vectors derived from filamentous bacteriophage such as m13, and the like. Suitable Streptomyces plasmids include p1J101, and streptomyces bacteriophages such as fC31. Bacillus plasmids include pC194, pC221, pT127, and the like. Suitable Pseudomonas plasmids have been reviewed by Izaki (Jpn. J. Bacteriol. 33:729-742, 1978) and John et al. (Rev. Infect. Dis. 8:693-704, 1986).

To express a protease of the invention (or a functional derivative thereof) in a prokaryotic cell, it is necessary to operably link the sequence encoding the protease of the invention to a functional prokaryotic promoter. Such promoters are either constitutive or inducible promoters, but commonly inducible promoters are used. Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (PL and PR), the trp, recA, lacZ, lacI, and gal promoters of E. coli, the α-amylase and the V-28-specific promoters of B. subtilis, the promoters of the bacteriophages of Bacillus, and Streptomyces promoters. Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984). Additionally, proper expression in a prokaryotic cell also requires the presence of a ribosome-binding site upstream of the encoding sequence. Such ribosome-binding sites are disclosed, for example, by Gold et al. (Ann. Rev. Microbiol. 35:365-404, 1981).

### Fusion Protein

Proteins may be expressed as fusion proteins. Genes for proteins expressed as fusion proteins ligated into expression vectors that add a number of amino acids to a protein encoded and expressed, usually to the amino terminus of the recombinant protein. Such a strategy of producing fusion proteins is usually adopted for three purposes: (1) to assist in the purification by acting as a ligand in affinity purification, (2) to increase the solubility of the product, and (3) to increase the expression of the product. Often, expression vectors for use in fusion protein production, a proteolytic cleavage site is included at the junction of the fusion region and the protein of interest to enable purification of the recombinant protein away from the fusion region following affinity purification of the fusion protein. Such enzymes, and their cognate recognition sequences, include Factor Xa, thrombin and enterokinase, and may also include trypsin or chymotrypsin. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

### Improving Yield

Maximizing recombinant protein expression in E. coli can be assisted by expressing the protein or fusion protein in host bacteria with an impaired proteolytic system so as to reduce the post-synthesis degradation of the recombinant protein (Gottesman, S., Gene Expression Technology: Methods in Enzymology 185, Academic Press, San Diego, Calif. (1990) 119-128). Another strategy is to alter the mix of codons used in the coding sequence to reflect the usage of the individual codons for each amino acid in the host (e.g., E. coli (Wada et al., (1992) Nucleic Acids Res. 20:2111-2118)). Such alteration of nucleic acid sequences of the invention can be carried out by standard DNA synthesis techniques and may prove useful for a variety of prokaryotic and eukaryotic expression systems.

### Eukaryotic Hosts

Suitable hosts may include eukaryotic cells. Preferred eukaryotic hosts include, for example, yeast, fungi, insect cells, and mammalian cells both in vivo and in tissue culture. Useful mammalian cell hosts include HeLa cells, cells of fibroblast origin such as VERO or CHO-K1, and cells of lymphoid origin and their derivatives. Preferred mammalian host cells include SP2/0 and J558L, as well as neuroblastoma cell lines such as IMR 332, which may provide better capacities for correct post-translational processing. In general, eukaryotic organisms such as yeast provide substantial advantages in that they can also carry out post-translational modifications.

A large number of yeast expression systems may be potentially utilized which incorporate promoter and termination elements from the actively expressed sequences coding for glycolytic enzymes. These expression systems produce in large quantities of proteins when yeast are grown in mediums rich in glucose. Known glycolytic gene sequences can also provide very efficient transcriptional control signals. A number of recombinant DNA strategies exist utilizing strong promoter sequences and high copy number plasmids which can be utilized for production of the desired proteins in yeast. Examples of vectors suitable for expression in S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (InVitrogen Corporation, San Diego, Calif.), and picZ (InVitrogen Corp, San Diego, Calif.).

In another embodiment, the protein of interest may be expressed in insect cells for example the Drosophila larvae. Using insect cells as hosts, the Drosophila alcohol dehydrogenase promoter may be used (Rubin, Science 240: 1453-1459, 1988). Additionally, baculovirus vectors can be engineered to express large amounts of the protein of interest in cultured insect cells (e.g., Sf 9 cells)(Jasny, Science 238:1653, 1987; Miller et al., in: Genetic Engineering, Vol. 8, Plenum, Setlow et al., eds., pp. 277-297, 1986). Vectors which may be used include the pAc series (Smith et al. (1983) Mol. Cell Biol. 3:2156-2165) and the pVL series (Lucklow and Summers (1989) Virology 170:31-39).

Plant cells may also be utilized as hosts, and control sequences compatible with plant cells are available, such as the cauliflower mosaic virus 35S and 19S promoters, and nopaline synthase promoter and polyadenylation signal sequences. Furthermore, the protein of interest may be expressed in plants which have incorporated the expression vector into their germ line.

In yet another embodiment, a nucleic acid of the invention may be expressed in mammalian cells using a mammalian expression vector. Possibilities and techniques for expression in mammalian cells has recently been summarized (Colosimo, et al., “Transfer and expression of foreign genes in mammalian cells,” Biotechniques 29(2):314-8, 320-2, 324 passim, 2000; which is hereby incorporated by reference in its entirety including any drawings, tables, and figures.). Examples of mammalian expression vectors include pCDM8 (Seed, B. (1987) Nature 329:840) and pMT2PC (Kaufinan et al. (1987) EMBO J. 6:187-195). For use in mammalian cells, the regulatory sequences of the expression vector are often derived from viral regulatory elements. For example, commonly used promoters are derived from Simian Virus 40 (SV40), polyoma, Adenovirus 2, and cytomegalovirus (CMV) viruses. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984). Alternatively, promoters from mammalian expression products, such as actin, collagen, myosin, and the like, may be employed. Regulatory elements may also be derived from adenovirus, bovine papilloma virus, cytomegalovirus, simian virus, or the like.

Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation. Expression of proteins of interest in eukaryotic hosts requires the use of eukaryotic regulatory regions. Such regions will, in general, include a promoter region sufficient to direct the initiation of RNA synthesis.

The recombinant mammalian expression vector may also be designed to be capable of directing expression of the nucleic acid preferentially in a particular cell type (i.e., tissue-specific regulatory elements are used to control the expression). Such tissue-specific promoters include the liver-specific albumin promoter (Pinkert et al. (1987) Genes Dev. 1:268-277); lymphoid-specific promoters (e.g., Calame and Eaton (1988) Adv. Immunol. 43:235-275), and in particular promoters of immunoglobulins and T cell receptors (Winoto and Baltimore (1989) EMBO J. 8:729-733, Banerji et al. (1983) Cell 33:729-740; Queen and Baltimore (1983) Cell 33:741-748); mammary gland-specific promoters (e.g., milk whey promoter; U.S. Pat. No. 4,873,316 and European Application Publication No. 264,166); and pancreas-specific promoters (Edlund et al. (1985) Science 230:912-916). Developmentally-regulated promoters may also be utilized, for example, the α-fetoprotein promoter (Campes and Tilghman (1989) Genes Dev. 3:537-546), and the murine hox promoters (Kessel and Gruss (1990) Science 249:374-379).

Preferred eukaryotic plasmids include, for example, SV40, BPV, pMAM-neo, pKRC, vaccinia, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance,” Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, N.Y., pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, DEAE-dextran-mediated transfection, lipofection, calcium phosphate-precipitation, direct microinjection, and the like. Suitable methods for transforming or transfecting host cells can be found in Sambrook, et al. (2001). After the introduction of the vector, recipient cells are grown in a selective medium, which selects for the growth of vector-containing cells. Expression of the cloned gene(s) results in the production of a protein of interest, or fragments thereof.

For other suitable expression systems for both prokaryotic and eukaryotic cells see Sambrook, et al., “Molecular Cloning: A Laboratory Manual,” 3rd ed., Cold Spring Harbor Laboratory, Cold Spring Harbor Laboratory Press, Cold Spring Harbor, N.Y., 2001, which is hereby incorporated by reference in its entirety, including any drawings, figures, and tables.

For transformation of eukaryotic cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, neomycin, methotrexate, glyphosate, and bialophos. Nucleic acid encoding a selectable marker can be introduced into a host cell on the same vector as that encoding the protein of interest or can be introduced on a separate vector. Cells stably transformed with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the protein of interest. Accordingly, the invention further provides methods for producing the protein of interest using the host cells of the invention. In one embodiment, the method comprises culturing the host cell into which a recombinant expression vector encoding the protein of interest has been introduced in a suitable medium such that the protein of interest is produced, and may be purified by one skilled in the art.

In many embodiments, the IP-10 analogs are designed using one or more of the following: (1) replacing all or part of the beta-sheet domain of an IP-10 chemokine with a linker; (2) connecting all or a portion of the amino-terminal domain and all or a portion of the carboxy-terminal domain of an IP-10 chemokine with a linker; (3) cyclizing residues within an analog; (4) replacing the cysteines of an IP-10 chemokine with other amino acids; and (5) attaching modifying groups to an IP-10 analog such as, for example, to the amino terminus, carboxy terminus, or a combination thereof.

Modifying Groups

Generally speaking, the IP-10 mimetics may include derivatives of IP-10 chemokines, analogs of IP-10 chemokines, and conservatively modified variants thereof. Examples of such mimetics include C-terminal hydroxymethyl derivatives; O-modified derivatives (e.g., C-terminal hydroxymethyl benzyl ether); N-terminally modified derivatives including substituted amides such as alkylamides and hydrazides and compounds in which a C-terminal phenylalanine residue is replaced with a phenethylamide analogue (e.g., Ser-Ile-phenethylamide as an analog of the tripeptide Ser-Ile-Phe); glycosylated IP-10 chemokine derivatives; polyethylene glycol modified derivatives; biotinylated derivatives; and combinations thereof The IP-10 analogs may also include a pharmaceutically acceptable salt of an IP-10 analog.

In some embodiments, the IP-10 analogs may be coupled directly or indirectly to at least one modifying group. The term “modifying group” can be used in some embodiments to describe structures that are directly attached to the peptidic structure (e.g., by covalent bonding or covalent coupling), as well as those that are indirectly attached to the peptidic structure (e.g., by a stable non-covalent bond association or by covalent coupling through a linker to additional amino acid residues). The term “modifying group” may also refer to mimetics, analogues or derivatives thereof, which may flank the IP-10 core peptidic structure.

In some embodiments, the modifying groups can be coupled to the amino-terminus or carboxy-terminus of an IP-10 peptidic structure, or to a peptidic or peptidomimetic region flanking the core structure. Alternatively, the modifying group can be coupled to a side chain of at least one amino acid residue of an IP-10 peptidic structure, or to a peptidic or peptido-mimetic region flanking the core domain (e.g., through the epsilon amino group of a lysyl residue(s); through the carboxyl group of an aspartic acid residue(s) or a glutamic acid residue(s); through a hydroxy group of a tyrosyl residue(s), a serine residue(s) or a threonine residue(s); or any other suitable reactive group on an amino acid side chain). The modifying groups can be covalently coupled to the peptidic structure and can be attached by means and methods well known in the art for linking chemical structures, including, for example, amide, alkylamino, sulfide, carbamate or urea bonds.

In some embodiments, the modifying group may comprise a cyclic, heterocyclic or polycyclic group. The term “cyclic group,” as used herein, includes cyclic saturated or unsaturated (i.e., aromatic) group having from 3 to 10; from 4 to 8; or 5, 6, or 7 carbon atoms. Exemplary non-aromatic cyclic groups include cyclopropyl, cyclobutyl, cyclopentyl, cyclohexyl, and cyclooctyl groups. The term “heterocyclic group” includes optionally substituted, saturated or unsaturated, three- to eight-membered cyclic structures in which one or more skeletal atoms is oxygen, nitrogen, sulfur, or combinations thereof.

Cyclic groups may be unsubstituted or substituted at one or more ring positions. A cyclic group may for example be substituted with halogens, alkyls, cycloalkyls, alkenyls, alkynyls, aryls, arylalkyls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN. The cyclic group may also be linked to a substituent, such as halogens, alkyls, cycloalkyls, alkenyls, alkynyls, aryls, arylalkyls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, or —CN, by means of a saturated or unsaturated chain of 1, 2, 3, 4, 5, 6, 7, 8, or more carbon atoms; additionally, one or more of the carbon atoms may be replaced with an oxygen, nitrogen, or sulfur atom. One of skill will appreciate that these means of linking are taught for example only, and that other means of linking are available in the art.

The term “heterocyclic group” includes cyclic saturated, unsaturated and aromatic groups having from 3 to 10; from 4 to 8; or 5, 6, or 7 carbon atoms, wherein the ring structure includes about one or more heteroatoms. Heterocyclic groups include pyrrolidine, oxolane, thiolane, imidazole, oxazole, piperidine, piperazine, morpholine. The heterocyclic ring may be substituted at one or more positions with such substituents as, for example, halogens, alkyls, cycloalkyls, alkenyls, alkynyls, aryls, arylalkyls, other heterocycles, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN. Heterocycles may also be bridged or fused to other cyclic groups as described below. A linker may also link the heterocyclic group to such substituents as, for example, halogens, alkyls, cycloalkyls, alkenyls, alkynyls, aryls, arylalkyls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN.

The term “polycyclic group” as used herein is intended to refer to two or more saturated, unsaturated, or aromatic cyclic rings in which two or more carbons are common to two adjoining rings, so that the rings are “fused rings.” Rings that are joined through non-adjacent atoms are termed “bridged” rings. Each of the rings of the polycyclic group may be substituted with such substituents as described above, as for example, halogens, alkyls, cycloalkyls, alkenyls, alkynyls, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, or —CN.

The term “alkyl” refers to saturated aliphatic groups, including straight chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl has 20 or fewer carbon atoms in its backbone (C₁-C₂₀ for straight chain, C₃-C₂₀ for branched chain), 15 or few carbon atoms; 12 or fewer carbon atoms; or 10 or fewer carbon atoms. In some embodiments, cycloalkyls may have from 4-10 carbon atoms in their ring structure, such as rings made from 5, 6 or 7 carbon atoms. Unless the number of carbons is otherwise specified, “lower alkyl” as used herein means an alkyl group, as defined above, having from one to ten carbon atoms in its backbone structure. Likewise, “lower alkenyl” and “lower alkynyl” have chain lengths of ten or less carbons.

The term “alkyl” (or “lower alkyl”) as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups)), and esters (including alkyloxycarbonyl and aryloxycarbonyl groups), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety.

The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), or silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF3, —CN, and the like.

The terms “alkenyl” and “alkynyl” refer to unsaturated aliphatic groups analogous in length and possible substitution to the alkyls described above, but that contain at least one double or triple bond respectively. The term “aralkyl,” as used herein, refers to an alkyl or alkylenyl group substituted with at least one aryl group. Exemplary aralkyls include benzyl (i.e., phenylmethyl), 2-naphthylethyl, 2-(2-pyridyl)propyl, 5-dibenzosuberyl, and the like. The term “alkylcarbonyl,” as used herein, refers to —C(O)-alkyl. Similarly, the term “arylcarbonyl” refers to —C(O)-aryl. The term “alkyloxycarbonyl,” as used herein, refers to the group —C(O)—O-alkyl, and the term “aryloxycarbonyl” refers to —C(O)—O-aryl. The term “acyloxy” refers to —O—C(O)—R₇, in which R₇ is alkyl, alkenyl, alkynyl, aryl, aralkyl or heterocyclyl.

The term “amino,” as used herein, refers to —N(R_{α})(R_{β}), in which R_{α} and R_{β} are each independently hydrogen, alkyl, alkyenyl, alkynyl, aralkyl, aryl, or in which R_{α} and R_{β} together with the nitrogen atom to which they are attached form a ring having 4-8 atoms. Thus, the term “amino,” as used herein, includes unsubstituted, monosubstituted (e.g., monoalkylamino or monoarylamino), and disubstituted (e.g., dialkylamino or alkylarylamino) amino groups. The term “amido” refers to —C(O)—N(R_{α})(R_{β}), in which R_{α} and R_{β} are as defined above. The term “acylamino” refers to —N(R′_{α})C(O)—R₇, in which R₇ is as defined above and R_{α} is alkyl. As used herein, the term “nitro” means —NO₂; the term “halogen” designates —F, —Cl, —Br or —I; the term “sulfhydryl” means —SH; and the term “hydroxyl” means —OH.

The term “aryl” as used herein includes 5-, 6- and 7-membered aromatic groups that may include from zero to four heteroatoms in the ring, for example, phenyl, pyrrolyl, furyl, thiophenyl, imidazolyl, oxazole, thiazolyl, triazolyl, pyrazolyl, pyridyl, pyrazinyl, pyridazinyl and pyrimidinyl, and the like. Those aryl groups having heteroatoms in the ring structure may also be referred to as “aryl heterocycles” or “heteroaromatics.” The aromatic ring can be substituted at one or more ring positions with such substituents as described above, as for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, a heterocyclyl, an aromatic or heteroaromatic moiety, —CF₃, —CN, or the like. Aryl groups can also be part of a polycyclic group. For example, aryl groups include fused aromatic moieties such as naphthyl, anthracenyl, quinolyl, indolyl, and the like.

In some embodiments, the molecular weight of a modifying group should be at or below about 40,000 Daltons. In some embodiments, the molecular weight ranges from about 100 Daltons to about 40,000 Daltons, from about 300 Daltons to about 30,000 Daltons, from about 200 Daltons to about 20,000 Daltons, from about 200 Daltons to about 10,000 Daltons, from about 300 Daltons to about 5,000 Daltons, from about 500 Daltons to about 500 Daltons, or any range therein. It is to be appreciated that one skilled in the art should recognize that some of the groups, subgroups, and individual biobeneficial agents may not be used in some embodiments of the present invention.

### Functional Enhancement

An IP-10 chemokine analog compound of the invention may be further modified to alter the specific properties of the compound while retaining the desired functionality of the compound. For example, in one embodiment, the compound may be modified to alter a pharmacokinetic property of the compound, such as in vivo stability, solubility, bioavailability or half-life. The compound may be modified to label the compound with a detectable substance. In addition, the compound may be modified to couple the compound to an additional therapeutic moiety.

To further chemically modify the compound, such as to alter its pharmacokinetic properties, reactive groups can be derivatized. For example, when the modifying group is attached to the amino-terminal end of the IP-10 core domain, the carboxy-terminal end of the compound may be further modified. Potential C-terminal modifications include those that reduce the ability of the compound to act as a substrate for carboxypeptidases. Examples of C-terminal modifiers include an amide group, an ethylamide group and various non-natural amino acids, such as D-amino acids, β-alanine, C-terminal decarboxylation, and a C-terminal alcohol. Alternatively, when the modifying group is attached to the carboxy-terminal end of the aggregation core domain, the amino-terminal end of the compound may be further modified, for example, to reduce the ability of the compound to act as a substrate for aminopeptidases.

IP-10 chemokines and IP-10 analogs of the invention may be modified by the addition of polyethylene glycol (PEG). PEG modification may lead to improved circulation time, improved solubility, improved resistance to proteolysis, reduced antigenicity and immunogenicity, improved bioavailability, reduced toxicity, improved stability, and easier formulation (For a review see, Francis et al., International Journal of Hematology 68:1-18, 1998). PEGylation may also result in a substantial reduction in bioactivity. In some embodiments, the molecular weight of the PEG can be 40,000 Daltons or less, 30,000 Daltons or less, 20,000 Daltons or less, 10,000 Daltons or less, 5000 Daltons or less, 3000 Daltons or less 1000 Daltons or less, or any range therein. In some embodiments, the molecular weight of the PEG can range from about 100 Daltons to about 20,000 Daltons, from about 200 Daltons to about 5,000 Daltons, from about 300 Daltons to about 3000 Daltons, from about 400 Daltons to about 2000 Daltons, from about 500 Daltons to about 1000 Daltons, or any range therein.

The IP-10 analogs of the invention may also be coupled to a radioisotope such as yttrium-90, F-18 or iodine-131 for therapeutic purposes (see, e.g., DeNardo et al., “Choosing an optimal radioimmunotherapy dose for clinical response,” Cancer 94(4 Suppl): 1275-86, 2002; Kaltsas et al., “The value of radiolabelled MIBG and octreotide in the diagnosis and management of neuroendocrine tumours,” Ann Oncol 12 Suppl 2:S47-50, 2001).

An IP-10 chemokine mimetic compound can be further modified to label the compound by combining the compound with a detectable substance. In some aspects of the invention, suitable detectable substances include various enzymes, prosthetic groups, fluorescent materials, luminescent materials, light scattering or plasmon resonant materials, and radioactive materials. Modifying groups may also include groups comprising biochemical labels or structures, such as biotin, a diethylene-triaminepentaacetyl group, a (O)-menthoxyacetyl group, a N-acetylneuraminyl group, a cholyl structure or an iminobiotinyl group.

Examples of suitable enzymes include horseradish peroxidase, alkaline phosphatase, beta-galactosidase, or acetylcholinesterase. Examples of suitable prosthetic groups which are members of a binding pair and are capable of forming complexes include streptavidin/biotin, avidin/biotin and an antigen/antibody complex (e.g., rabbit IgG and anti-rabbit IgG).

Examples of suitable fluorescent materials include umbelliferone, fluorescein, fluorescein isothiocyanate, rhodamine, dichlorotriazinylamine fluorescein, dansyl chloride or phycoerythrin and energy transfer fluorescent dyes. An example of a luminescent material includes luminol. Examples of light scattering or plasmon resonant materials include gold or silver particles and quantum dots. Examples of suitable radioactive material include ¹⁴C, ¹²³I, ¹²⁴I, ¹²⁵I, ¹³¹I, Tc99m, ³⁵S or ³H. A chemokine mimetic compound may be radioactively labeled with ¹⁴C, either by incorporation of ¹⁴C into the modifying group or one or more amino acid structures in the chemokine mimetic compound.

Labeled chemokine mimetic compounds may be used to assess the in vivo pharmacokinetics of the compounds, as well as to detect disease progression or propensity of a subject to develop a disease, for example for diagnostic purposes. Tissue distribution can be detected using a labeled chemokine mimetic compound either in vivo or in an in vitro sample derived from a subject. A modifying group can be chosen that provides a site at which a chelation group for the label can be introduced, such as the Aic derivative of cholic acid, which has a free amino group. For example, a tyrosine residue within IP-10 sequence may be substituted with radioactive iodotyrosyl. Any of the various isotopes of radioactive iodine may be incorporated to create a diagnostic or therapeutic agent. For example, a chemokine mimetic compound of the invention may be labeled with radioactive technetium or iodine for use as a diagnostic agent: ¹²³I (half-life=13.2 hours) may be used for whole body scintigraphy, ¹²⁴I (half life=4 days) may be used for positron emission tomography (PET), ¹²⁵I (half life=60 days) may be used for metabolic turnover studies and ¹³¹I (half life=8 days) may be used for whole body counting and delayed low resolution imaging studies.

In some embodiments, the IP-10 analog may be modified at its carboxy terminus with a cholyl group according to methods known in the art. Cholyl derivatives and analogs may also be used as modifying groups. For example, a preferred cholyl derivative is Aic (3-(O-aminoethyl-iso)-cholyl), which has a free amino group that can be used to further modify the chemokine mimetic compound. A modifying group may be a “biotinyl structure,” which includes biotinyl groups and analogues and derivatives thereof (such as a 2-iminobiotinyl group).

In some embodiments, the modifying group may comprise a fluorescent-label group, e.g., a fluorescein-containing group, such as a group derived from reacting an IP-10 derived peptidic structure with 5-(and 6-)-carboxyfluorescein, succinimidyl ester or fluorescein isothiocyanate. The IP-10 analogs may also be modified by attaching other fluorescent labels including rhodamine, dichlorotriazinylamine fluorescein, dansyl chloride or phycoerythrin and energy transfer fluorescent dyes or fluorescent ion indicators.

In various embodiments, the modifying group(s) may comprise an N-acetylneuraminyl group, a trans-4-cotininecarboxyl group, a 2-imino-1-imidazolidineacetyl group, an (S)-(-)-indoline-2-carboxyl group, a (-)-menthoxyacetyl group, a 2-norbornaneacetyl group, a γ-oxo-5-acenaphthenebutyryl, a (-)-2-oxo-4-thiazolidinecarboxyl group, a tetrahydro-3-furoyl group, a 2-iminobiotinyl group, a diethylenetriaminepentaacetyl group, a 4-morpholinecarbonyl group, a 2-thiopheneacetyl group or a 2-thiophenesulfonyl group.

In some embodiments, light scattering groups, magnetic groups, nanogold, peptides, proteins, a solid matrix, radiolabels, or carbohydrates may be attached. In some embodiments, the modifying group may be an oligomer such as, for example, an oligonucleotide, or an oligopeptide (which may or may not be derived from an IP-10 chemokine). In some embodiments, the modifying group can include a polysaccharide, a glycosaminoglycan, a poly(alkylene glycol), or a combination thereof.

In some embodiments, the modifying group can be an RDG or RGD peptide, a hydrolyzable chemical moiety for a prodrug form of the IP-10 analog, or another therapeutic agent connected to the IP-10 analog through a hydrolysable chemical moiety to form a codrug form of the IP-10 analog. The prodrugs and codrugs are capable of being transformed, upon metabolism in vivo, into an IP-10 analog agonist or antagonist. Such a form of a modifying group can be referred to as a “secondary modifying group.” A variety of strategies are known in the art for preparing peptide prodrugs and codrugs that limit metabolism in order to optimize delivery of the active form of the peptide-based drug.

IP-10 Sequence Listines:

Using the teachings provided herein, one of skill can create a variety of IP-10 analogs. Preferred embodiments of linear IP-10 analogs of the present invention corresponding to a portion of N-terminal have the following structures, wherein the underlined residues are cyclized, and in some embodiments the cyclization may include, for example, formation of a disulphide bond, an alkenyl bond, an ether bond, or a lactam; and, any additional amino acids identified in square brackets in a header indicates that the peptide described by the header is at least an analog of a native fragment, and the position of the amino acid substitutions are noted by the standard abbreviation for the amino acid substituent with a superscript number indicating the residue positions at which the modification has occurred, corresponding to the native sequence:

IP-10-(1-14) acid or amide
(SEQ ID NO:163)
b1) RNH-Val-Pro-Leu-Ser-Arg-Thr-Val-Arg-Cys-Thr-Cys-Ile-Ser-Ile-(OH)NH₂
(SEQ ID NO:164)
b2) RNH-Val-Pro-Xaa₃-Ser-Arg-Thr-Val-Arg-Xaa₁-Thr-Xaa₂-Ile-Ser-Ile-(OH)NH₂
(SEQ ID NO:165)
b3) RNH-Val-Pro-Leu-Xaa₃-Arg-Thr-Val-Arg-Xaa₁-Thr-Xaa₂-Ile-Ser-Ile-(OH)NH₂
(SEQ ID NO:166)
b4) RNH-Val-Pro-Leu-Ser-Xaa₃-Thr-Val-Arg-Xaa₁-Thr-Xaa₂-Ile-Ser-Ile-(OH)NH₂
(SEQ ID NO:167)
b5) RNH-Val-Pro-Leu-Ser-Arg-Xaa₃-Val-Arg-Xaa₁-Thr-Xaa₂-Ile-Ser-Ile-(OH)NH₂
(SEQ ID NO:168)
b6) RNH-Val-Pro-Leu-Ser-Arg-Thr-Xaa₃-Arg-Xaa₁-Thr-Xaa₂-Ile-Ser-Ile-(OH)NH₂
(SEQ ID NO:169)
b7) RNH-Val-Pro-Leu-Ser-Arg-Thr-VaI-Arg-Xaa₃-Xaa₁-Thr-Xaa₂-lle-Ser-Ile-
(OH)NH₂
IP-10-(1-17) acid or amide
(SEQ ID NO:170)
b8) RNH-Val-Pro-Leu-Ser-Arg-Thr-Val-Arg-Cys-Thr-Cys-Ile-Ser-Ile-Ser-Asn-Gln-
(OH)NH₂
(SEQ ID NO:171)
b9) RNH-Val-Pro-Xaa₃-Ser-Arg-Thr-Val-Arg-Xaa₁-Thr-Xaa₂-Ile-Ser-Ile-Ser-Asn-
Gln-(OH)NH₂
(SEQ ID NO:172)
b10) RNH-Val-Pro-Leu-Xaa₃-Arg-Thr-Val-Arg-Xaa₁-Thr-Xaa₂-Ile-Ser-Ile-Ser-Asn-
Gln-(OH)NH₂
(SEQ ID NO:173)
b11) RNH-Val-Pro-Leu-Ser-Xaa₃-Thr-Val-Arg-Xaa₁-Thr-Xaa₂-Ile-Ser-Ile-Ser-Asn-
Gln-(OH)NH₂
(SEQ ID NO:174)
b12) RNH-Val-Pro-Leu-Ser-Arg-Xaa₃-Val-Arg-Xaa₁-Thr-Xaa₂-Ile-Ser-Ile-Ser-Asn-
Gln-(OH)NH₂
(SEQ ID NO:175)
b13) RNH-Val-Pro-Leu-Ser-Arg-Thr-Xaa₃-Arg-Xaa₁-Thr-Xaa₂-Ile-Ser-Ile-Ser-Asn-
Gln-(OH)NH₂
(SEQ ID NO:176)
b14) RNH-Val-Pro-Leu-Ser-Arg-Thr-Val-Arg-Xaa₃-Xaa₁-Thr-Xaa₂-Ile-Ser-Ile-Ser-
Asn-Gln-(OH)NH₂

Preferred embodiments of linear IP-10 analogs of the present invention corresponding to a portion of the internal region of IP-10 having the following structures:

[A¹¹]-IP-10-(11-35) acid or amide
(SEQ ID NO:177)
b15) RNH-Ala-Ile-Ser-Ile-Ser-Asn-Gln-Pro-Val-Asn-Pro-Arg-Ser-Leu-Glu-Lys-Leu-
Glu-Ile-Ile-Pro-Ala-Ser-Gln-Phe-(OH)NH₂

Preferred embodiments of linear IP-10 analogs of the present invention corresponding to a portion of the N-terminal region and the internal region of IP-10 having the following structures:

IP-10-(1-35) acid or amide
(SEQ ID NO:178)
b16) RNH-Val-Pro-Leu-Ser-Arg-Thr-Val-Arg-Cys-Thr-Cys-Ile-Ser-Ile-Ser-Asn-Gln-
Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:179)
b17) RNH-Xaa₃-Pro-Leu-Ser-Arg-Thr-Val-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:180)
b18) RNH-Val-Xaa₃-Leu-Ser-Arg-Thr-Val-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:181)
b19) RNH-Val-Pro-Xaa₃-Ser-Arg-Thr-Val-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:182)
b20) RNH-Val-Pro-Leu-Xaa₃-Arg-Thr-Val-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:183)
b21) RNH-Val-Pro-Leu-Ser-Xaa₃-Thr-Val-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:184)
b22) RNH-Val-Pro-Leu-Ser-Arg-Xaa₃-Val-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:185)
b23) RNH-Val-Pro-Leu-Ser-Arg-Thr-Xaa₃-Arg-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂
(SEQ ID NO:186)
b24) RNH-Val-Pro-Leu-Ser-Arg-Thr-Val-Xaa₃-Xaa₁-Thr-Xaa₂-Ile-Ser-Ile-Ser-Asn-
Gln-Pro-Val-Asn-Pro-Arg-Ser-Leu-Glu-Lys-Leu-Glu-Ile-Ile-Pro-Ala-Ser-Gln-Phe-
(OH)NH₂

Preferred embodiments of linear IP-10 analogs of the present invention corresponding to a portion of the C-terminal region of IP-10 having the following sequence:

IP-10-(53-77) acid or amide
(SEQ ID NO:187)
b25) RNH-Leu-Asn-Pro-Glu-Ser-Lys-Ala-Ile-Lys-Asn-Leu-Leu-Lys-Ala-Val-Ser-
Lys-Glu-Met-Ser-Lys-Arg-Ser-Pro-(OH)NH₂

Preferred embodiments of cyclic IP-10 analogs of the present invention corresponding to a portion of the N-terminal region joined with a linker to a cyclic portion of the C-terminal region of IP-10 having the following structures:

IP-10-(1-14)-[linker]-IP-10-(65-77)-acid or amide
(SEQ ID NO:188)
b26) RNH-Val-Pro-Leu-Ser-Arg-Thr-Val-Arg-Cys-Thr-Cys-Ile-Ser-Ile-[linker]-Leu-
Lys-Ala-Val-Ser-Lys-Glu-Met-Ser-Lys-Arg-Ser-Pro-(OH)NH₂
(SEQ ID NO:189)
b27) RNH-Xaa₃-Pro-Leu-Ser-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:190)
b28) RNH-Val-Xaa₃-Leu-Ser-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:191)
b29) RNH-Val-Pro-Xaa₃-Ser-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:192)
b30) RNH-Val-Pro-Leu-Xaa₃-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:193)
b31) RNH-Val-Pro-Leu-Ser-Xaa₃-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:194)
b32) RNH-Val-Pro-Leu-Ser-Arg-Xaa₃-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:195)
b33) RNH-Val-Pro-Leu-Ser-Arg-Thr-Xaa₃-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:196)
b34) RNH-Val-Pro-Leu-Ser-Arg-Thr-Val-Xaa₃-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:197)
b35) RNH-Val-Pro-Leu-Ser-Arg-Thr-Val-Arg-Cys-Thr-Cys-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:198)
b36) RNH-Xaa₃-Pro-Leu-Ser-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:199)
b37) RNH-Val-Xaa₃-Leu-Ser-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:200)
b38) RNH-Val-Pro-Xaa₃-Ser-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:201)
b39) RNH-Val-Pro-Leu-Xaa₃-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:202)
b40) RNH-Val-Pro-Leu-Ser-Xaa₃-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:203)
b41) RNH-Val-Pro-Leu-Ser-Arg-Xaa₃-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:204)
b42) RNH-Val-Pro-Leu-Ser-Arg-Thr-Xaa₃-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:205)
b43) RNH-Val-Pro-Leu-Ser-Arg-Thr-Val-Xaa₃-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:206)
b44) RNH-Val-Pro-Leu-Ser-Arg-Thr-Val-Arg-Cys-Thr-Cys-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:207)
b45) RNH-Xaa₃-Pro-Leu-Ser-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:208)
b46) RNH-Val-Xaa₃-Leu-Ser-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:209)
b47) RNH-Val-Pro-Xaa₃-Ser-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:210)
b48) RNH-Val-Pro-Leu-Xaa₃-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:211)
b49) RNH-Val-Pro-Leu-Ser-Xaa₃-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:212)
b50) RNH-Val-Pro-Leu-Ser-Arg-Xaa₃-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:213)
b51) RNH-Val-Pro-leu-Ser-Arg-Thr-Xaa₃-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:214)
b52) RNH-Val-Pro-Leu-Ser-Arg-Thr-Val-Xaa₃-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:215)
b53) RNH-Val-Pro-Leu-Ser-Arg-Thr-Val-Arg-Cys-Thr-Cys-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:216)
b54) RNH-Xaa₃-Pro-Leu-Ser-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:217)
b55) RNH-Val-Xaa₃-Leu-Ser-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:218)
b56) RNH-Val-Pro-Xaa₃-Ser-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:219)
b57) RNH-Val-Pro-Leu-Xaa₃-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:220)
b58) RNH-Val-Pro-Leu-Ser-Xaa₃-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:221)
b59) RNH-Val-Pro-Leu-Ser-Arg-Xaa₃-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:222)
b60) RNH-Val-Pro-Leu-Ser-Arg-Thr-Xaa₃-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:223)
b61) RNH-Val-Pro-Leu-Ser-Arg-Thr-Val-Xaa₃-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
IP-10-(1-14)-[linker]-IP-10-(54-66)-acid or amide
(SEQ ID NO:224)
b62) RNH-Val-Pro-Leu-Ser-Arg-Thr-Val-Arg-Cys-Thr-Cys-Ile-Ser-Ile-[linker]-Leu-
Asn-Pro-Glu-Ser-Lys-Ala-Ile-Lys-Asn-Leu-Leu-Lys-(OH)NH₂
(SEQ ID NO:225)
b63) RNH-Xaa₃-Pro-Leu-Ser-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:226)
b64) RNH-Val-Xaa₃-Leu-Ser-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:227)
b65) RNH-Val-Pro-Xaa₃-Ser-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NIH2
(SEQ ID NO:228)
b66) RNH-Val-Pro-Leu-Xaa₃-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:229)
b67) RNH-Val-Pro-Leu-Ser-Xaa₃-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:230)
b68) RNH-Val-Pro-Leu-Ser-Arg-Xaa₃-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:231)
b69) RNH-Val-Pro-Leu-Ser-Arg-Thr-Xaa₃-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:232)
b70) RNH-Val-Pro-Leu-Ser-Arg-Thr-Val-Xaa₃-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:233)
b71) RNH-Val-Pro-Leu-Ser-Arg-Thr-Val-Arg-Cys-Thr-Cys-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:234)
b72) RNH-Xaa₃-Pro-Leu-Ser-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:235)
b73) RNH-Val-Xaa₃-Leu-Ser-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:236)
b74) RNH-Val-Pro-Xaa₃-Ser-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:237)
b75) RNH-Val-Pro-Leu-Xaa₃-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:238)
b76) RNH-Val-Pro-Leu-Ser-Xaa₃-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:239)
b77) RNH-Val-Pro-Leu-Ser-Arg-Xaa₃-Val-Arg-Xaa₁-Thr-Xaa₂-Xaa₁-Ser-Ile-[linker]-
Leu-Asn-Pro-Glu-Ser-Lys-Ala-Ile-Lys-Asn-Leu-Leu-Lys-(OH)NH₂
(SEQ ID NO:240)
b78) RNH-Val-Pro-Leu-Ser-Arg-Thr-Xaa₃-Arg-Xaa₁-Thr-Xaa₂-Xaa₁-Ser-Ile-[linker]-
Leu-Asn-Pro-Glu-Ser-Lys-Ala-Ile-Lys-Asn-Leu-Leu-Lys-(OH)NH₂
(SEQ ID NO:241)
b79) RNH-Val-Pro-Leu-Ser-Arg-Thr-Val-Xaa₃-Xaa₁-Thr-Xaa₂-Xaa₁-Ser-Ile-[linker]-
Leu-Asn-Pro-Glu-Ser-Lys-Ala-Ile-Lys-Asn-Leu-Leu-Lys-(OH)NH₂
(SEQ ID NO:242)
b80) RNH-Val-Pro-Leu-Ser-Arg-Thr-Val-Arg-Cys-Thr-Cys-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:243)
b81) RNH-Xaa₃-Pro-Leu-Ser-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:244)
b82) RNH-Val-Xaa₃-Leu-Ser-Arg-Thr-Val-Arg-Xaa₁-Thr-Xaa₂-Ile-Xaa₁-Ile-[linker]-
Leu-Asn-Pro-Glu-Ser-Lys-Ala-Ile-Lys-Asn-Leu-Leu-Lys-(OH)NH₂
(SEQ ID NO:245)
b83) RNH-Val-Pro-Xaa₃-Ser-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:246)
b84) RNH-Val-Pro-Leu-Xaa₃-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:247)
b85) RNH-Val-Pro-Leu-Ser-Xaa₃-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:248)
b86) RNH-Val-Pro-Leu-Ser-Arg-Xaa₃-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:249)
b87) RNH-Val-Pro-Leu-Ser-Arg-Thr-Xaa₃-Arg-Xaa₁-Thr-Xaa₂-Ile-Xaa₁-Ile-[linker]-
Leu-Asn-Pro-Glu-Ser-Lys-Ala-Ile-Lys-Asn-Leu-Leu-Lys-(OH)NH₂
(SEQ ID NO:250)
b88) RNH-Val-Pro-Leu-Ser-Arg-Thr-Val-Xaa₃-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:251)
b89) RNH-Val-Pro-Leu-Ser-Arg-Thr-Val-Arg-Cys-Thr-Cys-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:252)
b90) RNH-Xaa₃-Pro-Leu-Ser-Arg-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:253)
b91) RNH-Val-Xaa₃-Leu-Ser-Arg-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:254)
b92) RNH-Val-Pro-Xaa₃-Ser-Arg-Thr-Val-Arg-Xaa₁-Thr-Xaa₂-Ile-Ser-Xaa₄-[linker]-
Leu-Asn-Pro-Glu-Ser-Lys-Ala-IIe-Lys-Asn-Leu-Leu-Lys-(OH)NH₂
(SEQ ID NO:255)
b93) RNH-Val-Pro-Leu-Xaa₃-Arg-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:256)
b94) RNH-Val-Pro-Leu-Ser-Xaa₃-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:257)
b95) RNH-Val-Pro-Leu-Ser-Arg-Xaa₃-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:258)
b96) RNH-Val-Pro-Leu-Ser-Arg-Thr-Xaa₃-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:259)
b97) RNH-Val-Pro-Leu-Ser-Arg-Thr-Val-Xaa₃-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
IP-10-(1-14)-[linker]-IP-10-(59-71)-acid or amide
(SEQ ID NO:260)
b98) RNH-Val-Pro-Leu-Ser-Arg-Thr-Val-Arg-Cys-Thr-Cys-Ile-Ser-Ile-[linker]-
Lys-Ala-Ile-Lys-Asn-Leu-Leu-Lys-Ala-Val-Ser-Lys-Glu-(OH)NH₂
(SEQ ID NO:261)
b99) RNH-Xaa₃-Pro-Leu-Ser-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:262)
b100) RNH-Val-Xaa₃-Leu-Ser-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:263)
b101) RNH-Val-Pro-Xaa₃-Ser-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:264)
b102) RNH-Val-Pro-Leu-Xaa₃-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:265)
b103) RNH-Val-Pro-Leu-Ser-Xaa₃-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:266)
b104) RNH-Val-Pro-Leu-Ser-Arg-Xaa₃-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:267)
b105) RNH-Val-Pro-Leu-Ser-Arg-Thr-Xaa₃-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:268)
b106) RNH-Val-Pro-Leu-Ser-Arg-Thr-Val-Xaa₃-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:269)
b107) RNH-Val-Pro-Leu-Ser-Arg-Thr-Val-Arg-Cys-Thr-Cys-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:270)
b108) RNH-Xaa₃-Pro-Leu-Ser-Arg-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:271)
b109) RNH-Val-Xaa₃-Leu-Ser-Arg-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:272)
b110) RNH-Val-Pro-Xaa₃-Ser-Arg-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:273)
b111) RNH-Val-Pro-Leu-Xaa₃-Arg-hr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:274)
b112) RNH-Val-Pro-Leu-Ser-Xaa₃-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:275)
b113) RNH-Val-Pro-Leu-Ser-Arg-Xaa₃-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:276)
b114) RNH-Val-Pro-Leu-Ser-Arg-Thr-Xaa₃-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:277)
b115) RNH-Val-Pro-Leu-Ser-Arg-Thr-Val-Xaa₃-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:278)
b116) RNH-Val-Pro-Leu-Ser-Arg-Thr-Val-Arg-Cys-Thr-Cys-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:279)
b117) RNH-Xaa₃-Pro-Leu-Ser-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:280)
b118) RNH-Val-Xaa₃-Leu-Ser-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:281)
b119) RNH-Val-Pro-Xaa₃-Ser-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:282)
b120) RNH-Val-Pro-Leu-Xaa₃-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:283)
b121) RNH-Val-Pro-Leu-Ser-Xaa₃-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:284)
b122) RNH-Val-Pro-Leu-Ser-Arg-Xaa₃-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:285)
b123) RNH-Val-Pro-Leu-Ser-Arg-Thr-Xaa₃-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:286)
b124) RNH-Val-Pro-Leu-Ser-Arg-Thr-Val-Xaa₃-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:287)
b125) RNH-Val-Pro-Leu-Ser-Arg-Thr-Val-Arg-Cys-Thr-Cys-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:288)
b126) RNH-Xaa₃-Pro-Leu-Ser-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:289)
b127) RNH-Val-Xaa₃-Leu-Ser-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:290)
b128) RNH-Val-Pro-Xaa₃-Ser-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:291)
b129) RNH-Val-Pro-Leu-Xaa₃-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:292)
b130) RNH-Val-Pro-Leu-Ser-Xaa₃-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:293)
b131) RNH-Val-Pro-Leu-Ser-Arg-Xaa₃-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:294)
b132) RNH-Val-Pro-Leu-Ser-Arg-Thr-Xaa₃-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:295)
b133) RNH-Val-Pro-Leu-Ser-Arg-Thr-Val-Xaa₃-Xaa₁-Thr-Xaa₂-IIe-Ser-Xaa₄-[linker]-
Lys-Ala-Ile-Lys-Asn-Leu-Leu-Lys-Ala-Val-Ser-Lys-GIu-(OH)NH₂
IP-10-(1-17)-[linker]-IP-10-(65-77)-acid or amide
(SEQ ID NO:296)
b134) RNH-Val-Pro-Leu-Ser-Arg-Thr-Val-Arg-Cys-Thr-Cys-Ile-Ser-Ile-Ser-Asn-Gln-
[linker]-Leu-Lys-Ala-Val-Ser-Lys-Glu-Met-Ser-Lys-Arg-Ser-Pro-(OH)NH₂
(SEQ ID NO:297)
b135) RNH-Xaa₃-Pro-Leu-Ser-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:298)
b136) RNH-Val-Xaa₃-Leu-Ser-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:299)
b137) RNH-Val-Pro-Xaa₃-Ser-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:300)
b138) RNH-Val-Pro-Leu-Xaa₃-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:301)
b139) RNH-Val-Pro-Leu-Ser-Xaa₃-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:302)
b140) RNH-Val-Pro-Leu-Ser-Arg-Xaa₃-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:303)
b141) RNH-Val-Pro-Leu-Ser-Arg-Thr-Xaa₃-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:304)
b142) RNH-Val-Pro-Leu-Ser-Arg-Thr-Val-Xaa₃-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:305)
b143) RNH-Val-Pro-Leu-Ser-Arg-Thr-Val-Arg-Cys-Thr-Cys-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:306)
b144) RNH-Xaa₃-Pro-Leu-Ser-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:307)
b145) RNH-Val-Xaa₃-Leu-Ser-Arg-Thr-Val-Arg-Xaa₁-Thr-Xaa₂-Xaa₁-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:308)
b146) RNH-Val-Pro-Xaa₃-Ser-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:309)
b147) RNH-Val-Pro-Leu-Xaa₃-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:310)
b148) RNH-Val-Pro-Leu-Ser-Xaa₃-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:311)
b149) RNH-Val-Pro-Leu-Ser-Arg-Xaa₃-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:312)
b150) RNH-Val-Pro-Leu-Ser-Arg-Thr-Xaa₃-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:313)
b151) RNH-Val-Pro-Leu-Ser-Arg-Thr-Val-Xaa₃-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:314)
b152) RNH-Val-Pro-Leu-Ser-Arg-Thr-Val-Arg-Cys-Thr-Cys-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:315)
b153) RNH-Xaa₃-Pro-Leu-Ser-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:316)
b154) RNH-Val-Xaa₃-Leu-Ser-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:317)
b155) RNH-Val-Pro-Xaa₃-Ser-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:318)
b156) RNH-Val-Pro-Leu-Xaa₃-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:319)
b157) RNH-Val-Pro-Leu-Ser-Xaa₃-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:320)
b158) RNH-Val-Pro-Leu-Ser-Arg-Xaa₃-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:321)
b159) RNH-Val-Pro-Leu-Ser-Arg-Thr-Xaa₃-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:322)
b160) RNH-Val-Pro-Leu-Ser-Arg-Thr-Val-Xaa₃-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:323)
b161) RNH-Val-Pro-Leu-Ser-Arg-Thr-Val-Arg-Cys-Thr-Cys-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:324)
b162) RNH-Xaa₃-Pro-Leu-Ser-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:325)
b163) RNH-Val-Xaa₃-Leu-Ser-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:326)
b164) RNH-Val-Pro-Xaa₃-Ser-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:327)
b165) RNH-Val-Pro-Leu-Xaa₃-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:328)
b166) RNH-Val-Pro-Leu-Ser-Xaa₃-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:329)
b167) RNH-Val-Pro-Leu-Ser-Arg-Xaa₃-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:330)
b168) RNH-Val-Pro-Leu-Ser-Arg-Thr-Xaa₃-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:331)
b169) RNH-Val-Pro-Leu-Ser-Arg-Thr-Val-Xaa₃-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:332)
b170) RNH-Val-Pro-Leu-Ser-Arg-Thr-Val-Arg-Cys-Thr-Cys-Ile-Ser-Ile-Ser-Xaa₄-Gln-
[linker]-Leu-Lys-Ala-Val-Ser-Lys-Glu-Met-Ser-Lys-Arg-Ser-Pro-(OH)NH₂
(SEQ ID NO:333)
b171) RNH-Xaa₃-Pro-Leu-Ser-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:334)
b172) RNH-Val-Xaa₃-Leu-Ser-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:335)
b173) RNH-Val-Pro-Xaa₃-Ser-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:336)
b174) RNH-Val-Pro-Leu-Xaa₃-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:337)
b175) RNH-Val-Pro-Leu-Ser-Xaa₃-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:338)
b176) RNH-Val-Pro-Leu-Ser-Arg-Xaa₃-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:339)
b177) RNH-Val-Pro-Leu-Ser-Arg-Thr-Xaa₃-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:340)
b178) RNH-Val-Pro-Leu-Ser-Arg-Thr-Val-Xaa₃-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:341)
b179) RNH-Val-Pro-Leu-Ser-Arg-Thr-Val-Arg-Cys-Thr-Cys-Ile-Ser-Ile-Ser-Gln-Xaa₄-
[linker]-Leu-Lys-Ala-Val-Ser-Lys-Glu-Met-Ser-Lys-Arg-Ser-Pro-(OH)NH₂
(SEQ ID NO:342)
b180) RNH-Xaa₃-Pro-Leu-Ser-Arg-Thr-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:343)
b181) RNH-Val-Xaa₃-Leu-Ser-Arg-Thr-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:344)
b182) RNH-Val-Pro-Xaa₃-Ser-Arg-Thr-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:345)
b183) RNH-Val-Pro-Leu-Xaa₃-Arg-Thr-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:346)
b184) RNH-Val-Pro-Leu-Ser-Xaa₃-Thr-Val-Arg-Xaa₁-Thr-Xaa2-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:347)
b185) RNH-Val-Pro-Leu-Ser-Arg-Xaa₃-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:348)
b186) RNH-Val-Pro-Leu-Ser-Arg-Thr-Xaa₃-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:349)
b187) RNH-Val-Pro-Leu-Ser-Arg-Thr-Val-Xaa₃-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
IP-10-(1-17)-[linker]-IP-10-(54-66)-acid or amide
(SEQ ID NO:350)
b188) RNH-Val-Pro-Leu-Ser-Arg-Thr-Val-Arg-Cys-Thr-Cys-Ile-Ser-Ile-Ser-Asn-Gln-
[linker]-Leu-Asn-Pro-Glu-Ser-Lys-Ala-Ile-Lys-Asn-Leu-Leu-Lys-(OH)NH₂
(SEQ ID NO:351)
b189) RNH-Xaa₃-Pro-Leu-Ser-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:352)
b190) RNH-Val-Xaa₃-Leu-Ser-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:353)
b191) RNH-Val-Pro-Xaa₃-Ser-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:354)
b192) RNH-Val-Pro-Leu-Xaa₃-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:355)
b193) RNH-Val-Pro-Leu-Ser-Xaa₃-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:356)
b194) RNH-Val-Pro-Leu-Ser-Arg-Xaa₃-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:357)
b195) RNH-Val-Pro-Leu-Ser-Arg-Thr-Xaa₃-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:358)
b196) RNH-Val-Pro-Leu-Ser-Arg-Thr-Val-Xaa₃-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:359)
b197) RNH-Val-Pro-Leu-Ser-Arg-Thr-Val-Arg-Cys-Thr-Cys-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:360)
b198) RNH-Xaa₃-Pro-Leu-Ser-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:361)
b199) RNH-Val-Xaa₃-Leu-Ser-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:362)
b200) RNH-Val-Pro-Xaa₃-Ser-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:363)
b201) RNH-Val-Pro-Leu-Xaa₃-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:364)
b202) RNH-Val-Pro-Leu-Ser-Xaa₃-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:365)
b203) RNH-Val-Pro-Leu-Ser-Arg-Xaa₃-Val-Arg-Xaa₁-Thr-Xaa₂-Xaa₁-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:366)
b204) RNH-Val-Pro-Leu-Ser-Arg-Thr-Xaa₃-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:367)
b205) RNH-Val-Pro-Leu-Ser-Arg-Thr-Val-Xaa₃-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:368)
b206) RNH-Val-Pro-Leu-Ser-Arg-Thr-Val-Arg-Cys-Thr-Cys-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:369)
b207) RNH-Xaa₃-Pro-Leu-Ser-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:370)
b208) RNH-Val-Xaa₃-Leu-Ser-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:371)
b209) RNH-Val-Pro-Xaa₃-Ser-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:372)
b210) RNH-Val-Pro-Leu-Xaa₃-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:373)
b211) RNH-Val-Pro-Leu-Ser-Xaa₃-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:374)
b212) RNH-Val-Pro-Leu-Ser-Arg-Xaa₃-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:375)
b213) RNH-Val-Pro-Leu-Ser-Arg-Thr-Xaa₃-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:376)
b214) RNH-Val-Pro-Leu-Ser-Arg-Thr-Val-Xaa₃-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:377)
b215) RNH-Val-Pro-Leu-Ser-Arg-Thr-Val-Arg-Cys-Thr-Cys-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:378)
b216) RNH-Xaa₃-Pro-Leu-Ser-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:379)
b217) RNH-Val-Xaa₃-Leu-Ser-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:380)
b218) RNH-Val-Pro-Xaa₃-Ser-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:381)
b219) RNH-Val-Pro-Leu-Xaa₃-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:382)
b220) RNH-Val-Pro-Leu-Ser-Xaa₃-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:383)
b221) RNH-Val-Pro-Leu-Ser-Arg-Xaa₃-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:384)
b222) RNH-Val-Pro-Leu-Ser-Arg-Thr-Xaa₃-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:385)
b223) RNH-Val-Pro-Leu-Ser-Arg-Thr-Val-Xaa₃-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:386)
b224) RNH-Val-Pro-Leu-Ser-Arg-Thr-Val-Arg-Cys-Thr-Cys-Ile-Ser-Ile-Ser-Xaa₄-Gln-
[linker]-Leu-Asn-Pro-Glu-Ser-Lys-Ala-Ile-Lys-Asn-Leu-Leu-Lys-(OH)NH₂
(SEQ ID NO:387)
b225) RNH-Xaa₃-Pro-Leu-Ser-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:388)
b226) RNH-Val-Xaa₃-Leu-Ser-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:389)
b227) RNH-Val-Pro-Xaa₃-Ser-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:390)
b228) RNH-Val-Pro-Leu-Xaa₃-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:391)
b229) RNH-Val-Pro-Leu-Ser-Xaa₃-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:392)
b230) RNH-Val-Pro-Leu-Ser-Arg-Xaa₃-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:393)
b231) RNH-Val-Pro-Leu-Ser-Arg-Thr-Xaa₃-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:394)
b232) RNH-Val-Pro-Leu-Ser-Arg-Thr-Val-Xaa₃-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:395)
b233) RNH-Val-Pro-Leu-Ser-Arg-Thr-Val-Arg-Cys-Thr-Cys-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:396)
b234) RNH-Xaa₃-Pro-Leu-Ser-Arg-Thr-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:397)
b235) RNH-Val-Xaa₃-Leu-Ser-Arg-Thr-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:398)
b236) RNH-Val-Pro-Xaa₃-Ser-Arg-Thr-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:399)
b237) RNH-Val-Pro-Leu-Xaa₃-Arg-Thr-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:400)
b238) RNH-Val-Pro-Leu-Ser-Xaa₃-Thr-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:401)
b239) RNH-Val-Pro-Leu-Ser-Arg-Xaa₃-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:402)
b240) RNH-Val-Pro-Leu-Ser-Arg-Thr-Xaa₃-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:403)
b241) RNH-Val-Pro-Leu-Ser-Arg-Thr-Val-Xaa₃-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
IP-10-(1-17)-[linker]-IP-10-(59-71)-acid or amide
(SEQ ID NO:404)
b242) RNH-Val-Pro-Leu-Ser-Arg-Thr-Val-Arg-Cys-Thr-Cys-Ile-Ser-Ile-Ser-Asn-Gln-
[linker]-Lys-Ala-Ile-Lys-Asn-Leu-Leu-Lys-Ala-Val-Ser-Lys-Glu-(OH)NH₂
(SEQ ID NO:405)
b243) RNH-Xaa₃-Pro-Leu-Ser-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:406)
b244) RNH-Val-Xaa₃-Leu-Ser-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:407)
b245) RNH-Val-Pro-Xaa₃-Ser-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:408)
b246) RNH-Val-Pro-Leu-Xaa₃-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:409)
b247) RNH-Val-Pro-Leu-Ser-Xaa₃-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:410)
b248) RNH-Val-Pro-Leu-Ser-Arg-Xaa₃-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:411)
b249) RNH-Val-Pro-Leu-Ser-Arg-Thr-Xaa₃-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:412)
b250) RNH-Val-Pro-Leu-Ser-Arg-Thr-Val-Xaa₃-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:413)
b251) RNH-Val-Pro-Leu-Ser-Arg-Thr-Val-Arg-Cys-Thr-Cys-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:414)
b252) RNH-Xaa₃-Pro-Leu-Ser-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:415)
b253) RNH-Val-Xaa₃-Leu-Ser-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:416)
b254) RNH-Val-Pro-Xaa₃-Ser-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:417)
b255) RNH-Val-Pro-Leu-Xaa₃-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:418)
b256) RNH-Val-Pro-Leu-Ser-Xaa₃-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:419)
b257) RNH-Val-Pro-Leu-Ser-Arg-Xaa₃-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:420)
b258) RNH-Val-Pro-Leu-Ser-Arg-Thr-Xaa₃-Arg-Xaa₁-Thr-Xaa₂-Xaa₁-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:421)
b259) RNH-Val-Pro-Leu-Ser-Arg-Thr-Val-Xaa₃-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:422)
b260) RNH-Val-Pro-Leu-Ser-Arg-Thr-Val-Arg-Cys-Thr-Cys-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:423)
b261) RNH-Xaa₃-Pro-Leu-Ser-Arg-Thr-Val-Arg-Xaa₁-Thr-Xaa₂-Ile-Xaa₁-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:424)
b262) RNH-Val-Xaa₃-Leu-Ser-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:425)
b263) RNH-Val-Pro-Xaa₃-Ser-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:426)
b264) RNH-Val-Pro-Leu-Xaa₃-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:427)
b265) RNH-Val-Pro-Leu-Ser-Xaa₃-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:428)
b266) RNH-Val-Pro-Leu-Ser-Arg-Xaa₃-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:429)
b267) RNH-Val-Pro-Leu-Ser-Arg-Thr-Xaa₃-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:430)
b268) RNH-Val-Pro-Leu-Ser-Arg-Thr-Val-Xaa₃-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:431)
b269) RNH-Val-Pro-Leu-Ser-Arg-Thr-Val-Arg-Cys-Thr-Cys-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:432)
b270) RNH-Xaa₃-Pro-Leu-Ser-Arg-Thr-Val-Arg-Xaa₁-Thr-Xaa₂-Ile-Ser-Xaa₁-Ser-Asn-
Gln-[linker]-Lys-Ala-Ile-Lys-Asn-Leu-Leu-Lys-Ala-Val-Ser-Lys-Glu-(OH)NH₂
(SEQ ID NO:433)
b271) RNH-Val-Xaa₃-Leu-Ser-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:434)
b272) RNH-Val-Pro-Xaa₃-Ser-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:435)
b273) RNH-Val-Pro-Leu-Xaa₃-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:436)
b274) RNH-Val-Pro-Leu-Ser-Xaa₃-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:437)
b275) RNH-Val-Pro-Leu-Ser-Arg-Xaa₃-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:438)
b276) RNH-Val-Pro-Leu-Ser-Arg-Thr-Xaa₃-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:439)
b277) RNH-Val-Pro-Leu-Ser-Arg-Thr-Val-Xaa₃-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:440)
b278) RNH-Val-Pro-Leu-Ser-Arg-Thr-Val-Arg-Cys-Thr-Cys-Ile-Ser-Ile-Ser-Xaa₄-Gln-
[linker]-Lys-Ala-Ile-Lys-Asn-Leu-Leu-Lys-Ala-Val-Ser-Lys-Glu-(OH)NH₂
(SEQ ID NO:441)
b279) RNH-Xaa₃-Pro-Leu-Ser-Arg-Thr-Val-Arg-Xaa₁-Thr-Xaa₂-Ile-Ser-Ile-Ser-Xaa₁-
Gln-[linker]-Lys-Ala-Ile-Lys-Asn-Leu-Leu-Lys-Ala-Val-Ser-Lys-Glu-(OH)NH₂
(SEQ ID NO:442)
b280) RNH-Val-Xaa₃-Leu-Ser-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:443)
b281) RNH-Val-Pro-Xaa₃-Ser-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:444)
b282) RNH-Val-Pro-Leu-Xaa₃-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:445)
b283) RNH-Val-Pro-Leu-Ser-Xaa₃-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:446)
b284) RNH-Val-Pro-Leu-Ser-Arg-Xaa₃-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:447)
b285) RNH-Val-Pro-Leu-Ser-Arg-Thr-Xaa₃-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:448)
b286) RNH-Val-Pro-Leu-Ser-Arg-Thr-Val-Xaa₃-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:449)
b287) RNH-Val-Pro-Leu-Ser-Arg-Thr-Val-Arg-Cys-Thr-Cys-Ile-Ser-Ile-Ser-Gln-Xaa₄-
[linker]-Lys-Ala-Ile-Lys-Asn-Leu-Leu-Lys-Ala-Val-Ser-Lys-Glu-(OH)NH₂
(SEQ ID NO:450)
b288) RNH-Xaa₃-Pro-Leu-Ser-Arg-Thr-Val-Arg-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:451)
b289) RNH-Val-Xaa₃-Leu-Ser-Arg-Thr-Val-Arg-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:452)
b290) RNH-Val-Pro-Xaa₃-Ser-Arg-Thr-Val-Arg-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:453)
b291) RNH-Val-Pro-Leu-Xaa₃-Arg-Thr-Val-Arg-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:454)
b292) RNH-Val-Pro-Leu-Ser-Xaa₃-Thr-Val-Arg-Xaa₁-Thr-Xaa₂-Ile-Ser-Ile-Ser-Gln-
Xaa₁-[linker]-Lys-Ala-Ile-Lys-Asn-Leu-Leu-Lys-Ala-Val-Ser-Lys-Glu-(OH)NH₂
(SEQ ID NO:455)
b293) RNH-Val-Pro-Leu-Ser-Arg-Xaa₃-Val-Arg-Xaa₁-Thr-Xaap₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:456)
b294) RNH-Val-Pro-Leu-Ser-Arg-Thr-Xaa₃-Arg-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:457)
b295) RNH-Val-Pro-Leu-Ser-Arg-Thr-Val-Xaa₃-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂

Preferred embodiments of cyclic IP-10 analogs of the present invention corresponding to a portion the N-terminal region joined with a linker to a cyclic portion of the C-terminal region of IP-10 having the following structures (underlined residues are cyclized):

IP-10-(1-14)-[linker]-IP-10-(65-77)-cyclic (Glu71-Lys74) acid or amide
(SEQ ID NO:458)
b296) RNH-Val-Pro-Leu-Ser-Arg-Thr-Val-Arg-Cys-Thr-Cys-Ile-Ser-Ile-[linker]-Leu-
Lys-Ala-Val-Ser-Lys-Glu-Met-Ser-Lys-Arg-Ser-Pro-(OH)NH₂
(SEQ ID NO:459)
b297) RNH-Xaa₃-Pro-Leu-Ser-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:460)
b298) RNH-Val-Xaa₃-Leu-Ser-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:461)
b299) RNH-Val-Pro-Xaa₃-Ser-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:462)
b300) RNH-Val-Pro-Leu-Xaa₃-Arg-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:463)
b301) RNH-Val-Pro-Leu-Ser-Xaa₃-Thr-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:464)
b302) RNH-Val-Pro-Leu-Ser-Arg-Xaa₃-Val-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:465)
b303) RNH-Val-Pro-Leu-Ser-Arg-Thr-Xaa₃-Arg-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:466)
b304) RNH-Val-Pro-Leu-Ser-Arg-Thr-Val-Xaa₃-Xaa₁-Thr-Xaa₂-Ile-Ser-Ile-[linker]-
Leu-Lys-Ala-Val-Ser-Lys-Glu-Met-Ser-Lys-Arg-Ser-Pro-(OH)NH₂
(SEQ ID NO:467)
b305) RNH-Val-Pro-Leu-Ser-Arg-Thr-Val-Arg-Cys-Thr-Cys-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:468)
b306) RNH-Xaa₃-Pro-Leu-Ser-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:469)
b307) RNH-Val-Xaa₃-Leu-Ser-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:470)
b308) RNH-Val-Pro-Xaa₃-Ser-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:471)
b309) RNH-Val-Pro-Leu-Xaa₃-Arg-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:472)
b310) RNH-Val-Pro-Leu-Ser-Xaa₃-Thr-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:473)
b311) RNH-Val-Pro-Leu-Ser-Arg-Xaa₃-Val-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:474)
b312) RNH-Val-Pro-Leu-Ser-Arg-Thr-Xaa₃-Arg-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:475)
b313) RNH-Val-Pro-Leu-Ser-Arg-Thr-Val-Xaa₃-Xaa₁-Thr-Xaa₂-Xaa₄-Ser-Ile-[linker]-
Leu-Lys-Ala-Val-Ser-Lys-Glu-Met-Ser-Lys-Arg-Ser-Pro-(OH)NH₂
(SEQ ID NO:476)
b314) RNH-Val-Pro-Leu-Ser-Arg-Thr-Val-Arg-Cys-Thr-Cys-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:477)
b315) RNH-Xaa₃-Pro-Leu-Ser-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:478)
b316) RNH-Val-Xaa₃-Leu-Ser-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:479)
b317) RNH-Val-Pro-Xaa₃-Ser-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:480)
b318) RNH-Val-Pro-Leu-Xaa₃-Arg-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:481)
b319) RNH-Val-Pro-Leu-Ser-Xaa₃-Thr-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:482)
b320) RNH-Val-Pro-Leu-Ser-Arg-Xaa₃-Val-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:483)
b321) RNH-Val-Pro-Leu-Ser-Arg-Thr-Xaa₃-Arg-Xaa₁-Thr-Xaa₂-Ile-Xaa₄-Ile-[linker]-
Leu-Lys-Ala-Val-Ser-Lys-Glu-Met-Ser-Lys-Arg-Ser-Pro-(OH)NH₂
(SEQ ID NO:484)
b322) RNH-Val-Pro-Leu-Ser-Arg-Thr-Val-Xaa₃-Xaa₁-Thr-Xaa₂-Ile-Xaa₄-Ile- [linker]-
Leu-Lys-Ala-Val-Ser-Lys-Glu-Met-Ser-Lys-Arg-Ser-Pro-(OH)NH₂
(SEQ ID NO:485)
b323) RNH-Val-Pro-Leu-Ser-Arg-Thr-Val-Arg-Cys-Tbr-Cys-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:486)
b324) RNH-Xaa₃-Pro-Leu-Ser-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:487)
b325) RNH-Val-Xaa₃-Leu-Ser-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:488)
b326) RNH-Val-Pro-Xaa₃-Ser-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:489)
b327) RNH-Val-Pro-Leu-Xaa₃-Arg-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:490)
b328) RNH-Val-Pro-Leu-Ser-Xaa₃-Thr-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:491)
b329) RNH-Val-Pro-Leu-Ser-Arg-Xaa₃-Val-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:492)
b330) RNH-Val-Pro-Leu-Ser-Arg-Thr-Xaa₃-Arg-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
(SEQ ID NO:493)
b331) RNH-Val-Pro-Leu-Ser-Arg-Thr-Val-Xaa₃-Xaa₁-Thr-Xaa₂-Ile-Ser-Xaa₄-[linker]-
Leu-Lys-Ala-Val-Ser-Lys-Glu-Met-Ser-Lys-Arg-Ser-Pro-(OH)NH₂
IP-10-(1-17)-[linker]-IP-10-(65-77)-cyclic (Glu71-Lys74) acid or amide
(SEQ ID NO:494)
b332) RNH-Val-Pro-Leu-Ser-Arg-Thr-Val-Arg-Cys-Thr-Cys-Ile-Ser-Ile-Ser-Asn-Gln-
[linker]-Leu-Lys-Ala-Val-Ser-Lys-Glu-Met-Ser-Lys-Arg-Ser-Pro-(OH)NH₂
(SEQ ID NO:495)
b333) RNH-Xaa₃-Pro-Leu-Ser-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:496)
b334) RNH-Val-Xaa₃-Leu-Ser-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:497)
b335) RNH-Val-Pro-Xaa₃-Ser-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:498)
b336) RNH-Val-Pro-Leu-Xaa₃-Arg-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:499)
b337) RNH-Val-Pro-Leu-Ser-Xaa₃-Thr-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:500)
b338) RNH-Val-Pro-Leu-Ser-Arg-Xaa₃-Val-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:501)
b339) RNH-Val-Pro-Leu-Ser-Arg-Thr-Xaa₃-Arg-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:502)
b340) RNH-Val-Pro-Leu-Ser-Arg-Thr-Val-Xaa₃-Xaa₁-Thr-Xaa₂-Ile-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:503)
b341) RNH-Val-Pro-Leu-Ser-Arg-Thr-Val-Arg-Cys-Thr-Cys-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:504)
b342) RNH-Xaa₃-Pro-Leu-Ser-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:505)
b343) RNH-Val-Xaa₃-Leu-Ser-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:506)
b344) RNH-Val-Pro-Xaa₃-Ser-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:507)
b345) RNR-Val-Pro-Leu-Xaa₃-Arg-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:508)
b346) RNH-Val-Pro-Leu-Ser-Xaa₃-Thr-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:509)
b347) RNH-Val-Pro-Leu-Ser-Arg-Xaa₃-Val-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:510)
b348) RNH-Val-Pro-Leu-Ser-Arg-Thr-Xaa₃-Arg-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:511)
b349) RNH-Val-Pro-Leu-Ser-Arg-Thr-Val-Xaa₃-Xaa₁-Thr-Xaa₂-Xaa₄-Ser-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:512)
b350) RNH-Val-Pro-Leu-Ser-Arg-Thr-Val-Arg-Cys-Thr-Cys-Ile-Xaa₄-Ile-Ser-Asn-
Gln- [linker]-Leu-Lys-Ala-Val-Ser-Lys-Glu-Met-Ser-Lys-Arg-Ser-Pro-(OH)NH₂
(SEQ ID NO:513)
b351) RNH-Xaa₃-Pro-Leu-Ser-Arg-Thr-Val-Arg-Xaa₁-Thr-Xaa₂-Ile-Xaa₁-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:514)
b352) RNH-Val-Xaa₃-Leu-Ser-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:515)
b353) RNH-Val-Pro-Xaa₃-Ser-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:516)
b354) RNH-Val-Pro-Leu-Xaa₃-Arg-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:517)
b355) RNH-Val-Pro-Leu-Ser-Xaa₃-Thr-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:518)
b356) RNH-Val-Pro-Leu-Ser-Arg-Xaa₃-Val-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:519)
b357) RNH-Val-Pro-Leu-Ser-Arg-Thr-Xaa₃-Arg-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:520)
b358) RNH-Val-Pro-Leu-Ser-Arg-Thr-Val-Xaa₃-Xaa₁-Thr-Xaa₂-Ile-Xaa₄-Ile-Ser-Asn-
Gln-[linker]-Leu-Lys-Ala-Val-Ser-Lys-Glu-Met-Ser-Lys-Arg-Ser-Pro-(OH)NH₂
(SEQ ID NO:521)
b359) RNH-Val-Pro-Leu-Ser-Arg-Thr-Val-Arg-Cys-Thr-Cys-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:522)
b360) RNH-Xaa₃-Pro-Leu-Ser-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:523)
b361) RNH-Val-Xaa₃-Leu-Ser-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:524)
b362) RNH-Val-Pro-Xaa₃-Ser-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:525)
b363) RNH-Val-Pro-Leu-Xaa₃-Arg-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:526)
b364) RNH-Val-Pro-Leu-Ser-Xaa₃-Thr-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:527)
b365) RNH-Val-Pro-Leu-Ser-Arg-Xaa₃-Val-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:528)
b366) RNH-Val-Pro-Leu-Ser-Arg-Thr-Xaa₃-Arg-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:529)
b367) RNH-Val-Pro-Leu-Ser-Arg-Thr-Val-Xaa₃-Xaa₁-Thr-Xaa₂-Ile-Ser-Xaa₄-Ser-Asn-
Gln-[linker]-Leu-Lys-Ala-Val-Ser-Lys-Glu-Met-Ser-Lys-Arg-Ser-Pro-(OH)NH₂
(SEQ ID NO:530)
b368) RNH-Val-Pro-Leu-Ser-Arg-Thr-Val-Arg-Cys-Thr-Cys-Ile-Ser-Ile-Ser-Xaa₄-Gln-
[linker]-Leu-Lys-Ala-Val-Ser-Lys-Glu-Met-Ser-Lys-Arg-Ser-Pro-(OH)NH₂
(SEQ ID NO:531)
b369) RNH-Xaa₃-Pro-Leu-Ser-Arg-Thr-Val-Arg-Xaa₁-Thr-Xaa₂-Ile-Ser-Ile-Ser-Xaa₁-
Gln-[linker]-Leu-Lys-Ala-Val-Ser-Lys-Glu-Met-Ser-Lys-Arg-Ser-Pro-(OH)NH₂
(SEQ ID NO:532)
b370) RNH-Val-Xaa₃-Leu-Ser-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:533)
b371) RNH-Val-Pro-Xaa₃-Ser-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:534)
b372) RNH-Val-Pro-Leu-Xaa₃-Arg-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:535)
b373) RNH-Val-Pro-Leu-Ser-Xaa₃-Thr-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:536)
b374) RNH-Val-Pro-Leu-Ser-Arg-Xaa₃-Val-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:537)
b375) RNH-Val-Pro-Leu-Ser-Arg-Thr-Xaa₃-Arg-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:538)
b376) RNH-Val-Pro-Leu-Ser-Arg-Thr-Val-Xaa₃-Xaa₁-Thr-Xaa₂-Ile-Ser-Ile-Ser-Xaa₄-
Gln-[linker]-Leu-Lys-Ala-Val-Ser-Lys-Glu-Met-Ser-Lys-Arg-Ser-Pro-(OH)NH₂
(SEQ ID NO:539)
b377) RNH-Val-Pro-Leu-Ser-Arg-Thr-Val-Arg-Cys-Thr-Cys-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:540)
b378) RNH-Xaa₃-Pro-Leu-Ser-Arg-Thr-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:541)
b379) RNH-Val-Xaa₃-Leu-Ser-Arg-Thr-Val-Arg-Xaa₁-Thr-Xaa₂-Ile-Ser-Ile-Ser-
GlnXaa₄-[linker]-Leu-Lys-Ala-Val-Ser-Lys-Glu-Met-Ser-Lys-Arg-Ser-Pro-(OH)NH₂
(SEQ ID NO:542)
b380) RNH-Val-Pro-Xaa₃-Ser-Arg-Thr-Val-Arg-Xaa₁-Thr-Xaa₂-Ile-Ser-Ile-Ser-
GlnXaa₄-[linker]-Leu-Lys-Ala-Val-Ser-Lys-Glu-Met-Ser-Lys-Arg-Ser-Pro-(OH)NH₂
(SEQ ID NO:543)
b381) RNH-Val-Pro-Leu-Xaa₃-Arg-Thr-Val-Arg-Xaa₁-Thr-Xaa₂-Ile-Ser-Ile-Ser-
GlnXaa₁-[linker]-Leu-Lys-Ala-Val-Ser-Lys-Glu-Met-Ser-Lys-Arg-Ser-Pro-(OH)NH₂
(SEQ ID NO:544)
b382) RNH-Val-Pro-Leu-Ser-Xaa₃-Thr-Val-Arg-Xaa₁-Thr-Xaa₂-Ile-Ser-Ile-Ser-
GlnXaa₁-[linker]-Leu-Lys-Ala-Val-Ser-Lys-Glu-Met-Ser-Lys-Arg-Ser-Pro-(OH)NH₂
(SEQ ID NO:545)
b383) RNH-Val-Pro-Leu-Ser-Arg-Xaa₃-Val-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:546)
b384) RNH-Val-Pro-Leu-Ser-Arg-Thr-Xaa₃-Arg-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
(SEQ ID NO:548)
b385) RNH-Val-Pro-Leu-Ser-Arg-Thr-Val-Xaa₃-Xaa₁-Thr-Xaa₂-Ile-Ser-Ile-Ser-Gln-
Xaa₄-[linker]-Leu-Lys-Ala-Val-Ser-Lys-Glu-Met-Ser-Lys-Arg-Ser-Pro-(OH)NH₂
IP-10-(1-14)-[linker]-IP-10-(54-66)-cyclic(Glu57-Lys62) acid or amide
(SEQ ID NO:549)
b386) RNH-Val-Pro-Leu-Ser-Arg-Thr-Val-Arg-Cys-Thr-Cys-Ile-Ser-Ile-[linker]-Leu-
Asn-Pro-Glu-Ser-Lys-Ala-Ile-Lys-Asn-Leu-Leu-Lys-(OH)NH₂
(SEQ ID NO:550)
b387) RNH-Xaa₃-Pro-Leu-Ser-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:551)
b388) RNH-Val-Xaa₃-Leu-Ser-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:552)
b389) RNH-Val-Pro-Xaa₃-Ser-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:553)
b390) RNH-Val-Pro-Leu-Xaa₃-Arg-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:554)
b391) RNH-Val-Pro-Leu-Ser-Xaa₃-Thr-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:555)
b392) RNH-Val-Pro-Leu-Ser-Arg-Xaa₃-Val-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:556)
b393) RNH-Val-Pro-Leu-Ser-Arg-Thr-Xaa₃-Arg-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:557)
b394) RNH-Val-Pro-Leu-Ser-Arg-Thr-Val-Xaa₃-Xaa₁-Thr-Xaa₂-Ile-Ser-Ile-[linker]-
Leu-Asn-Pro-Glu-Ser-Lys-Ala-Ile-Lys-Asn-Leu-Leu-Lys-(OH)NH₂
(SEQ ID NO:558)
b395) RNH-Val-Pro-Leu-Ser-Arg-Thr-Val-Arg-Cys-Thr-Cys-Xaa₄Ser-Ile-[linker]-
Leu-Asn-Pro-Glu-Ser-Lys-Ala-Ile-Lys-Asn-Leu-Leu-Lys-(OH)NH₂
(SEQ ID NO:559)
b396) RNH-Xaa₃-Pro-Leu-Ser-Arg-Thr-Val-Arg-Xaa₁-Thr-Xaa₂-Xaa₁-Ser-Ile-[linker]-
Leu-Asn-Pro-Glu-Ser-Lys-Ala-Ile-Lys-Asn-Leu-Leu-Lys-(OH)NH₂
(SEQ ID NO:560)
b397) RNH-Val-Xaa₃-Leu-Ser-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:561)
b398) RNH-Val-Pro-Xaa₃-Ser-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:562)
b399) RNH-Val-Pro-Leu-Xaa₃-Arg-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:563)
b400) RNH-Val-Pro-Leu-Ser-Xaa₃-Thr-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:564)
b401) RNH-Val-Pro-Leu-Ser-Arg-Xaa₃-Val-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:565)
b402) RNH-Val-Pro-Leu-Ser-Arg-Thr-Xaa₃-Arg-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:566)
b403) RNH-Val-Pro-Leu-Ser-Arg-Thr-Val-Xaa₃-Xaa₁-Thr-Xaa₂-Xaa₄-Ser-Ile-[linker]-
Leu-Asn-Pro-Glu-Ser-Lys-Ala-Ile-Lys-Asn-Leu-Leu-Lys-(OH)NH₂
(SEQ ID NO:567)
b404) RNH-Val-Pro-Leu-Ser-Arg-Thr-Val-Arg-Cys-Thr-Cys-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:568)
b405) RNH-Xaa₃-Pro-Leu-Ser-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:569)
b406) RNH-Val-Xaa₃-Leu-Ser-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:570)
b407) RNH-Val-Pro-Xaa₃-Ser-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:571)
b408) RNH-Val-Pro-Leu-Xaa₃-Arg-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:572)
b409) RNH-Val-Pro-Leu-Ser-Xaa₃-Thr-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:573)
b410) RNH-Val-Pro-Leu-Ser-Arg-Xaa₃-Val-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:574)
b411) RNH-Val-Pro-Leu-Ser-Arg-Thr-Xaa₃-Arg-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:575)
b412) RNH-Val-Pro-Leu-Ser-Arg-Thr-Val-Xaa₃-Xaa₁-Thr-Xaa₂-Ile-Xaa₄-Ile-[linker]-
Leu-Asn-Pro-Glu-Ser-Lys-Ala-Ile-Lys-Asn-Leu-Leu-Lys-(OH)NH₂
(SEQ ID NO:576)
b413) RNH-Val-Pro-Leu-Ser-Arg-Thr-Val-Arg-Cys-Thr-Cys-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:577)
b414) RNH-Xaa₃-Pro-Leu-Ser-Arg-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:578)
b415) RNH-Val-Xaa₃-Leu-Ser-Arg-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:579)
b416) RNH-Val-Pro-Xaa₃-Ser-Arg-Thr-Val-Arg-Xaa₁-Thr-Xaa₂-Ile-Ser-Xaa₁-[linker]-
Leu-Asn-Pro-Glu-Ser-Lys-Ala-Ile-Lys-Asn-Leu-Leu-Lys-(OH)NH₂
(SEQ ID NO:580)
b417) RNH-Val-Pro-Leu-Xaa₃-Arg-Thr-Val-Arg-Xaa₁-Thr-Xaa₂-Ile-Ser-Xaa₁-[linker]-
Leu-Asn-Pro-Glu-Ser-Lys-Ala-Ile-Lys-Asn-Leu-Leu-Lys-(OH)NH₂
(SEQ ID NO:581)
b418) RNH-Val-Pro-Leu-Ser-Xaa₃-Thr-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:582)
b419) RNH-Val-Pro-Leu-Ser-Arg-Xaa₃-Val-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:583)
b420) RNH-Val-Pro-Leu-Ser-Arg-Thr-Xaa₃-Arg-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
(SEQ ID NO:584)
b421) RNH-Val-Pro-Leu-Ser-Arg-Thr-Val-Xaa₃-Xaa₁-Thr-Xaa₂-Ile-Ser-Xaa₄-[linker]-
Leu-Asn-Pro-Glu-Ser-Lys-Ala-Ile-Lys-Asn-Leu-Leu-Lys-(OH)NH₂
IP-10-(1-17)-[linker]-IP-10-(54-66)-cyclic(Glu57-Lys62) acid or amide
(SEQ ID NO:585)
b422) RNH-Val-Pro-Leu-Ser-Arg-Thr-Val-Arg-Cys-Thr-Cys-Ile-Ser-Ile-Ser-Asn-Gln-
[linker]-Leu-Asn-Pro-Glu-Ser-Lys-Ala-Ile-Lys-Asn-Leu-Leu-Lys-(OH)NH₂
(SEQ ID NO:586)
b423) RNH-Xaa₃-Pro-Leu-Ser-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:587)
b424) RNH-Val-Xaa₃-Leu-Ser-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:588)
b425) RNH-Val-Pro-Xaa₃-Ser-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:589)
b426) RNH-Val-Pro-Leu-Xaa₃-Arg-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:590)
b427) RNH-Val-Pro-Leu-Ser-Xaa₃-Thr-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:591)
b428) RNH-Val-Pro-Leu-Ser-Arg-Xaa₃-Val-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:592)
b429) RNH-Val-Pro-Leu-Ser-Arg-Thr-Xaa₃-Arg-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:593)
b430) RNH-Val-Pro-Leu-Ser-Arg-Thr-Val-Xaa₃-Xaa₁-Thr-Xaa₂-Ile-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:594)
b431) RNH-Val-Pro-Leu-Ser-Arg-Thr-Val-Arg-Cys-Thr-Cys-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:595)
b432) RNH-Xaa₃-Pro-Leu-Ser-Arg-Thr-Val-Arg-Xaa₁-Thr-Xaa₂-Xaa₄-Ser-Ile-Ser-Asn-
Gln-[linker]-Leu-Asn-Pro-Glu-Lys-Ala-Ile-Lys-Asn-Leu-Leu-Lys-(OH)NH₂
(SEQ ID NO:596)
b433) RNH-Val-Xaa₃-Leu-Ser-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:597)
b434) RNH-Val-Pro-Xaa₃-Ser-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:598)
b435) RNH-Val-Pro-Leu-Xaa₃-Arg-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:599)
b436) RNH-Val-Pro-Leu-Ser-Xaa₃-Thr-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:600)
b437) RNH-Val-Pro-Leu-Ser-Arg-Xaa₃-Val-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:601)
b438) RNH-Val-Pro-Leu-Ser-Arg-Thr-Xaa₃-Arg-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:602)
b439) RNH-Val-Pro-Leu-Ser-Arg-Thr-Val-Xaa₃-Xaa₁-Thr-Xaa₂-Xaa₄-Ser-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:603)
b440) RNH-Val-Pro-Leu-Ser-Arg-Thr-Val-Arg-Cys-Thr-Cys-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:604)
b441) RNH-Xaa₃-Pro-Leu-Ser-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:605)
b442) RNH-Val-Xaa₃-Leu-Ser-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:606)
b443) RNH-Val-Pro-Xaa₃-Ser-Arg-Thr-Val-Arg-Xaa₁-Thr-Xaa₂-Ile-Xaa₁-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:607)
b444) RNH-Val-Pro-Leu-Xaa₃-Arg-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:608)
b445) RNH-Val-Pro-Leu-Ser-Xaa₃-Thr-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:609)
b446) RNH-Val-Pro-Leu-Ser-Arg-Xaa₃-Val-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:610)
b447) RNH-Val-Pro-Leu-Ser-Arg-Thr-Xaa₃-Arg-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:611)
b448) RNH-Val-Pro-Leu-Ser-Arg-Thr-Val-Xaa₃-Xaa₁-Thr-Xaa₂-Ile-Xaa₄-Ile-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:612)
b449) RNH-Val-Pro-Leu-Ser-Arg-Thr-Val-Arg-Cys-Thr-Cys-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:613)
b450) RNH-Xaa₃-Pro-Leu-Ser-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:614)
b451) RNH-Val-Xaa₃-Leu-Ser-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:615)
b452) RNH-Val-Pro-Xaa₃-Ser-Arg-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:616)
b453) RNH-Val-Pro-Leu-Xaa₃-Arg-Thr-Val-Arg-Xaa₁-Thr-Xaa₂-Ile-Ser-Xaa₁-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:617)
b454) RNH-Val-Pro-Leu-Ser-Xaa₃-Thr-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:618)
b455) RNH-Val-Pro-Leu-Ser-Arg-Xaa₃-Val-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:619)
b456) RNH-Val-Pro-Leu-Ser-Arg-Thr-Xaa₃-Arg-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:620)
b457) RNH-Val-Pro-Leu-Ser-Arg-Thr-Val-Xaa₃-Xaa₁-Thr-Xaa₂-Ile-Ser-Xaa₄-Ser-Asn-
Gln-[linker]-Leu-Asn-Pro-Glu-Ser-Lys-Ala-Ile-Lys-Asn-Leu-Leu-Lys-(OH)NH₂
(SEQ ID NO:621)
b458) RNH-Val-Pro-Leu-Ser-Arg-Thr-Val-Arg-Cys-Thr-Cys-Ile-Ser-Ile-Ser-Xaa₄-Gln-
[linker]-Leu-Asn-Pro-Glu-Ser-Lys-Ala-Ile-Lys-Asn-Leu-Leu-Lys-(OH)NH₂
(SEQ ID NO:622)
b459) RNH-Xaa₃-Pro-Leu-Ser-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:623)
b460) RNH-Val-Xaa₃-Leu-Ser-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:624)
b461) RNH-Val-Pro-Xaa₃-Ser-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:625)
b462) RNH-Val-Pro-Leu-Xaa₃-Arg-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:626)
b463) RNH-Val-Pro-Leu-Ser-Xaa₃-Thr-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:627)
b464) RNH-Val-Pro-Leu-Ser-Arg-Xaa₃-Val-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:628)
b465) RNH-Val-Pro-Leu-Ser-Arg-Thr-Xaa₃-Arg-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:629)
b466) RNH-Val-Pro-Leu-Ser-Arg-Thr-Val-Xaa₃-Xaa₁-Thr-Xaa₂-Ile-Ser-Ile-Ser-Xaa₄-
Gln-[linker]-Leu-Asn-Pro-Glu-Ser-Lys-Ala-Ile-Lys-Asn-Leu-Leu-Lys-(OH)NH₂
(SEQ ID NO:630)
b467) RNH-Val-Pro-Leu-Ser-Arg-Thr-Val-Arg-Cys-Thr-Cys-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:631)
b468) RNH-Xaa₃-Pro-Leu-Ser-Arg-Thr-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:632)
b469) RNH-Val-Xaa₃-Leu-Ser-Arg-Thr-Val-Arg-Xaa₁-Thr-Xaa₂-Ile-Ser-Ile-Ser-
GlnXaa₄-[linker]-Leu-Asn-Pro-Glu-Ser-Lys-Ala-Ile-Lys-Asn-Leu-Leu-Lys-(OH)NH₂
(SEQ ID NO:633)
b470) RNH-Val-Pro-Xaa₃-Ser-Arg-Thr-Val-Arg-Xaa₁-Thr-Xaa₂-Ile-Ser-Ile-Ser-
GlnXaa₄-[linker]-Leu-Asn-Pro-Glu-Ser-Lys-Ala-Ile-Lys-Asn-Leu-Leu-Lys-(OH)NH₂
(SEQ ID NO:634)
b471) RNH-Val-Pro-Leu-Xaa₃-Arg-Thr-Val-Arg-Xaa₁-Thr-Xaa₂-Ile-Ser-Ile-Ser-
GlnXaa₄-[linker]-Leu-Asn-Pro-Glu-Ser-Lys-Ala-Ile-Lys-Asn-Leu-Leu-Lys-(OH)NH₂
(SEQ ID NO:635)
b472) RNH-Val-Pro-Leu-Ser-Xaa₃-Thr-Val-Arg-Xaa₁-Thr-Xaa₂-Ile-Ser-Ile-Ser-
GlnXaa₄-[linker]-Leu-Asn-Pro-Glu-Ser-Lys-Ala-Ile-Lys-Asn-Leu-Leu-Lys-(OH)NH₂
(SEQ ID NO:636)
b473) RNH-Val-Pro-Leu-Ser-Arg-Xaa₃-Val-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:637)
b474) RNH-Val-Pro-Leu-Ser-Arg-Thr-Xaa₃-Arg-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂
(SEQ ID NO:638)
b475) RNH-Val-Pro-Leu-Ser-Arg-Thr-Val-Xaa₃-Xaa₁-Thr-Xaa₂-Ile-Ser-Ile-Ser-Gln-
Xaa₄-[linker]-Leu-Asn-Pro-Glu-Ser-Lys-Ala-Ile-Lys-Asn-Leu-Leu-Lys-(OH)NH₂

In some embodiments IP-10 analogs were cyclized by etherification between Lys66 and Ser69 (underlined residues are cyclized).

IP-10-(1-17)-[linker]-IP-10-(59-71)-cyclic(Lys66-Ser69) acid or amide
(SEQ ID NO:639)
b476) RNH-Val-Pro-Leu-Ser-Arg-Thr-Val-Arg-Cys-Thr-Cys-Ile-Ser-Ile-[linker]-Lys-
Ala-Ile-Lys-Asn-Leu-Leu-Lys-Ala-Val-Ser-Lys-Glu-(OH)NH₂
(SEQ ID NO:640)
b477) RNH-Xaa₃-Pro-Leu-Ser-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:641)
b478) RNH-Val-Xaa₃-Leu-Ser-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:642)
b479) RNH-Val-Pro-Xaa₃-Ser-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:643)
b480) RNH-Val-Pro-Leu-Xaa₃-Arg-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:644)
b481) RNH-Val-Pro-Leu-Ser-Xaa₃-Thr-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:645)
b482) RNH-Val-Pro-Leu-Ser-Arg-Xaa₃-Val-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:646)
b483) RNH-Val-Pro-Leu-Ser-Arg-Thr-Xaa₃-Arg-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:647)
b484) RNH-Val-Pro-Leu-Ser-Arg-Thr-Val-Xaa₃-Xaa₁-Thr-Xaa₂-Ile-Ser-Ile-[linker]-
Lys-Ala-Ile-Lys-Asn-Leu-Leu-Lys-Ala-Val-Ser-Lys-Glu-(OH)NH₂
(SEQ ID NO:648)
b485) RNK-Val-Pro-Leu-Ser-Arg-Thr-Val-Arg-Cys-Thr-Cys-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:649)
b486) RNH-Xaa₃-Pro-Leu-Ser-Arg-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:650)
b487) RNH-Val-Xaa₃-Leu-Ser-Arg-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:651)
b488) RNH-Val-Pro-Xaa₃-Ser-Arg-Thr-Val-Arg-Xaa₁-Thr-Xaa₂-Xaa₁-Ser-Ile-[linker]-
Lys-Ala-Ile-Lys-Asn-Leu-Leu-Lys-Ala-Val-Ser-Lys-Glu-(OH)NH₂
(SEQ ID NO:652)
b489) RNH-Val-Pro-Leu-Xaa₃-Arg-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:653)
b490) RNH-Val-Pro-Leu-Ser-Xaa₃-Thr-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:654)
b491) RNH-Val-Pro-Leu-Ser-Arg-Xaa₃-Val-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:655)
b492) RNH-Val-Pro-Leu-Ser-Arg-Thr-Xaa₃-Arg-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:656)
b493) RNH-Val-Pro-Leu-Ser-Arg-Thr-Val-Xaa₃-Xaa₁-Thr-Xaa₂-Xaa₄-Ser-Ile-[linker]-
Lys-Ala-Ile-Lys-Asn-Leu-Leu-Lys-Ala-Val-Ser-Lys-Glu-(OH)NH₂
(SEQ ID NO:657)
b494) RNH-Val-Pro-Leu-Ser-Arg-Thr-Val-Arg-Cys-Thr-Cys-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:658)
b495) RNH-Xaa₃-Pro-Leu-Ser-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:659)
b496) RNH-Val-Xaa₃-Leu-Ser-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:660)
b497) RNH-Val-Pro-Xaa₃-Ser-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:661)
b498) RNH-Val-Pro-Leu-Xaa₃-Arg-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:662)
b499) RNH-Val-Pro-Leu-Ser-Xaa₃-Thr-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:663)
b500) RNH-Val-Pro-Leu-Ser-Arg-Xaa₃-Val-Arg-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:664)
b501) RNH-Val-Pro-Leu-Ser-Arg-Thr-Xaa₃-Arg-Xaa₁-Thr-Xaa₂-Ile-Xaa₁-Ile-[linker]-
Lys-Ala-Ile-Lys-Asn-Leu-Leu-Lys-Ala-Val-Ser-Lys-Glu-(OH)NH₂
(SEQ ID NO:665)
b502) RNH-Val-Pro-Leu-Ser-Arg-Thr-Val-Xaa₃-Xaa₁-Thr-Xaa₂-Ile-Xaa₄-Ile-[linker]-
Lys-Ala-Ile-Lys-Asn-Leu-Leu-Lys-Ala-Val-Ser-Lys-Glu-(OH)NH₂
(SEQ ID NO:666)
b503) RNH-Val-Pro-Leu-Ser-Arg-Thr-Val-Arg-Cys-Thr-Cys-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:667)
b504) RNH-Xaa₃-Pro-Leu-Ser-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:668)
b505) RNH-Val-Xaa₃-Leu-Ser-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:669)
b506) RNH-Val-Pro-Xaa₃-Ser-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:670)
b507) RNH-Val-Pro-Leu-Xaa₃-Arg-Thr-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:671)
b508) RNH-Val-Pro-Leu-Ser-Xaa₃-Thr-Val-Arg-Xaa₁-Thr-Xaa₂-Ile-Ser-Xaa₄-[linker]-
Lys-Ala-Ile-Lys-Asn-Le1u-Leu-Lys-Ala-Val-Ser-Lys-Glu-(OH)NH2
(SEQ ID NO:672)
b509) RNH-Val-Pro-Leu-Ser-Arg-Xaa₃-Val-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:673)
b510) RNH-Val-Pro-Leu-Ser-Arg-Thr-Xaa₃-Arg-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
(SEQ ID NO:674)
b511) RNH-Val-Pro-Leu-Ser-Arg-Thr-Val-Xaa₃-Xaa₁-Thr-Xaa₂-Ile-Ser-Xaa₄-[linker]-
Lys-Ala-Ile-Lys-Asn-Leu-Leu-Lys-Ala-Val-Ser-Lys-Glu-(OH)NH₂
IP-10-(1-17)-[linker]-IP-10-(59-71)-cyclic(Lys66-Ser69) acid or amide
(SEQ ID NO:675)
b512) RNH-Val-Pro-Leu-Ser-Arg-Thr-Val-Arg-Cys-Thr-Cys-Ile-Ser-Ile-Ser-Asn-Gln-
[linker]-Lys-Ala-Ile-Lys-Asn-Leu-Leu-Lys-Ala-Val-Ser-Lys-Glu-(OH)NH₂
(SEQ ID NO:676)
b513) RNH-Xaa₃-Pro-Leu-Ser-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:677)
b514) RNH-Val-Xaa₃-Leu-Ser-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:678)
b515) RNH-Val-Pro-Xaa₃-Ser-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:679)
b516) RNH-Val-Pro-Leu-Xaa₃-Arg-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:680)
b517) RNH-Val-Pro-Leu-Ser-Xaa₃-Thr-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:681)
b518) RNH-Val-Pro-Leu-Ser-Arg-Xaa₃-Val-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:682)
b519) RNH-Val-Pro-Leu-Ser-Arg-Thr-Xaa₃-Arg-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:683)
b520) RNH-Val-Pro-Leu-Ser-Arg-Thr-Val-Xaa₃-Xaa₁-Thr-Xaa₂-Ile-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:684)
b521) RNH-Val-Pro-Leu-Ser-Arg-Thr-Val-Arg-Cys-Thr-Cys-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:685)
b522) RNH-Xaa₃-Pro-Leu-Ser-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:686)
b523) RNH-Val-Xaa₃-Leu-Ser-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:687)
b524) RNH-Val-Pro-Xaa₃-Ser-Arg-Thr-Val-Arg-Xaa₁-Thr-Xaa₂-Xaa₁-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:688)
b525) RNH-Val-Pro-Leu-Xaa₃-Arg-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:689)
b526) RNH-Val-Pro-Leu-Ser-Xaa₃-Thr-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:690)
b527) RNH-Val-Pro-Leu-Ser-Arg-Xaa₃-Val-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:691)
b528) RNH-Val-Pro-Leu-Ser-Arg-Thr-Xaa₃-Arg-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:692)
b529) RNH-Val-Pro-Leu-Ser-Arg-Thr-Val-Xaa₃-Xaa₁-Thr-Xaa₂-Xaa₄-Ser-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:693)
b530) RNH-Val-Pro-Leu-Ser-Arg-Thr-Val-Arg-Cys-Thr-Cys-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:694)
b531) RNH-Xaa₃-Pro-Leu-Ser-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:695)
b532) RNH-Val-Xaa₃-Leu-Ser-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:696)
b533) RNH-Val-Pro-Xaa₃-Ser-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:697)
b534) RNH-Val-Pro-Leu-Xaa₃-Arg-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:698)
b535) RNH-Val-Pro-Leu-Ser-Xaa₃-Thr-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:699)
b536) RNH-Val-Pro-Leu-Ser-Arg-Xaa₃-Val-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:700)
b537) RNH-Val-Pro-Leu-Ser-Arg-Thr-Xaa₃-Arg-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:701)
b538) RNH-Val-Pro-Leu-Ser-Arg-Thr-Val-Xaa₃-Xaa₁-Thr-Xaa₂-Ile-Xaa₄-Ile-Ser-Asn-
Gln-[linker]-Lys-Ala-Ile-Lys-Asn-Leu-Leu-Lys-Ala-Val-Ser-Lys-Glu-(OH)NH₂
(SEQ ID NO:702)
b539) RNH-Val-Pro-Leu-Ser-Arg-Thr-Val-Arg-Cys-Thr-Cys-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:703)
b540) RNH-Xaa₃-Pro-Leu-Ser-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:704)
b541) RNH-Val-Xaa₃-Leu-Ser-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:705)
b542) RNH-Val-Pro-Xaa₃-Ser-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:706)
b543) RNH-Val-Pro-Leu-Xaa₃-Arg-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:707)
b544) RNH-Val-Pro-Leu-Ser-Xaa₃-Thr-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:708)
b545) RNH-Val-Pro-Leu-Ser-Arg-Xaa₃-Val-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:709)
b546) RNH-Val-Pro-Leu-Ser-Arg-Thr-Xaa₃-Arg-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:710)
b548) RNH-Val-Pro-Leu-Ser-Arg-Thr-Val-Xaa₃-Xaa₁-Thr-Xaa₂-Ile-Ser-Xaa₄-Ser-Asn-
Gln-[linker]-Lys-Ala-Ile-Lys-Asn-Leu-Leu-Lys-Ala-Val-Ser-Lys-Glu-(OH)NH₂
(SEQ ID NO:711)
b549) RNH-Val-Pro-Leu-Ser-Arg-Thr-Val-Arg-Cys-Thr-Cys-Ile-Ser-Ile-Ser-Xaa₄-Gln-
[linker]-Lys-Ala-Ile-Lys-Asn-Leu-Leu-Lys-Ala-Val-Ser-Lys-Glu-(OH)NH₂
(SEQ ID NO:712)
b550) RNH-Xaa₃-Pro-Leu-Ser-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:713)
b551) RNH-Val-Xaa₃-Leu-Ser-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:714)
b552) RNH-Val-Pro-Xaa₃-Ser-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:715)
b553) RNH-Val-Pro-Leu-Xaa₃-Arg-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:716)
b554) RNH-Val-Pro-Leu-Ser-Xaa₃-Thr-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:717)
b555) RNH-Val-Pro-Leu-Ser-Arg-Xaa₃-Val-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:718)
b556) RNH-Val-Pro-Leu-Ser-Arg-Thr-Xaa₃-Arg-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:719)
b557) RNH-Val-Pro-Leu-Ser-Arg-Thr-Val-Xaa₃-Xaa₁-Thr-Xaa₂-Ile-Ser-Ile-Ser-Xaa₄-
Gln-[linker]-Lys-Ala-Ile-Lys-Asn-Leu-Leu-Lys-Ala-Val-Ser-Lys-Glu-(OH)NH₂
(SEQ ID NO:720)
b558) RNH-Val-Pro-Leu-Ser-Arg-Thr-Val-Arg-Cys-Thr-Cys-Ile-Ser-Ile-Ser-Gln-Xaa₄-
[linker]-Lys-Ala-Ile-Lys-Asn-Leu-Leu-Lys-Ala-Val-Ser-Lys-Glu-(OH)NH₂
(SEQ ID NO:721)
b559) RNH-Xaa₃-Pro-Leu-Ser-Arg-Thr-Val-Arg-Xaa₁-Thr-Xaa₂-Ile-Ser-Ile-Ser-Gln-
Xaa₄-[linker]-Lys-Ala-Ile-Lys-Asn-Leu-Leu-Lys-Ala-Val-Ser-Lys-Glu-(OH)NH₂
(SEQ ID NO:722)
b560) RNH-Val-Xaa₃-Leu-Ser-Arg-Thr-Val-Arg-Xaa₁-Thr-Xaa₂-Ile-Ser-Ile-Ser-
GlnXaa₄[linker]-Lys-Ala-Ile-Lys-Asn-Leu-Leu-Lys-Ala-Val-Ser-Lys-Glu-(OH)NH₂
(SEQ ID NO:723)
b561) RNH-Val-Pro-Xaa₃-Ser-Arg-Thr-Val-Arg-Xaa₁-Thr-Xaa₂-Ile-Ser-Ile-Ser-
GlnXaa₄[linker]-Lys-Ala-Ile-Lys-Asn-Leu-Leu-Lys-Ala-Val-Ser-Lys-Glu-(OH)NH₂
(SEQ ID NO:724)
b562) RNH-Val-Pro-Leu-Xaa₃-Arg-Thr-Val-Arg-Xaa₁-Thr-Xaa₂-Ile-Ser-Ile-Ser-
GlnXaa₄[linker]-Lys-Ala-Ile-Lys-Asn-Leu-Leu-Lys-Ala-Val-Ser-Lys-Glu-(OH)NH₂
(SEQ ID NO:725)
b563) RNH-Val-Pro-Leu-Ser-Xaa₃-Thr-Val-Arg-Xaa₁-Thr-Xaa₂-Ile-Ser-Ile-Ser-
GlnXaa₄[linker]-Lys-Ala-Ile-Lys-Asn-Leu-Leu-Lys-Ala-Val-Ser-Lys-Glu-(OH)NH₂
(SEQ ID NO:726)
b564) RNH-Val-Pro-Leu-Ser-Arg-Xaa₃-Val-Arg-Xaa₁-Thr-Xaa₂-Ile-Ser-Ile-Ser-Gln-
GlnXaa₄[linker]-Lys-Ala-Ile-Lys-Asn-Leu-Leu-Lys-Ala-Val-Ser-Lys-Glu-(OH)NH₂
(SEQ ID NO:727)
b565) RNH-Val-Pro-Leu-Ser-Arg-Thr-Xaa₃-Arg-Xaa₁-Thr-Xaa₂-Ile-Ser-Ile-Ser-Gln-
GlnXaa₄[linker]-Lys-Ala-Ile-Lys-Asn-Leu-Leu-Lys-Ala-Val-Ser-Lys-Glu-(OH)NH₂
(SEQ ID NO:728)
b566) RNH-Val-Pro-Leu-Ser-Arg-Thr-Val-Xaa₃-Xaa₁-Thr-Xaa₂-Ile-Ser-Ile-Ser-Gln-
GlnXaa₄[linker]-Lys-Ala-Ile-Lys-Asn-Leu-Leu-Lys-Ala-Val-Ser-Lys-Glu-(OH)NH₂

In the above structures:
R can be independently selected from substituted, unsubstituted, hetero-, straight-chained, branched, cyclic, saturated or unsaturated aliphatic radicals; or a substituted, unsubstituted, or hetero- aromatic radicals; PEG and derivatives thereof; and any other modifying group;
Xaa₁ is selected from the group consisting of any L- or D-natural amino acid and any non-natural amino acid.
Xaa₂ is selected from the group consisting of any L- or D-natural amino acid and any non-natural amino acid.
Xaa₃ is selected from the group consisting of L-Pro, D-Pro, P*, Btd and any L- or D-natural and non-natural amino acid.
Xaa₄ is selected from the group consisting of P*, Btd and any L- or D-natural amino acid and any non-natural amino acid.
P* is:

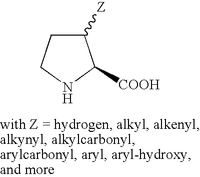

A wide variety of amino acid substitutions may be made in polypeptide sequences, such as lysine to glutamic acid, lysine to aspartic acid, Orn to Glu, Orn to Asp. Moieties other than naturally occurring amino acids may also be substituted, such as Btd:
Btd* is:

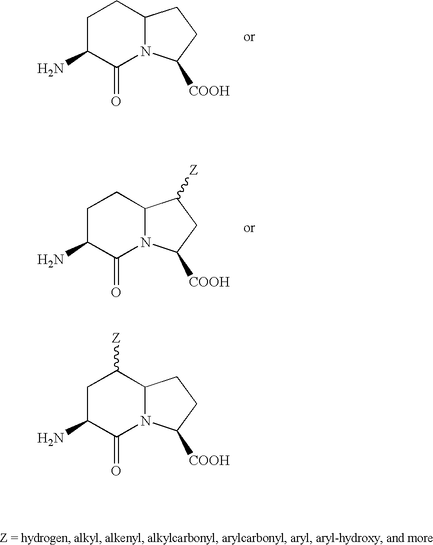

In some embodiments, R can be selected from the group consisting of hydrogen, alkyl, alkenyl, alkynyl, alkylcarbonyl, arylcarbonyl, aryl, PEG (polyethyleneglycol) and any other modifying group described herein. In some embodiments, R can comprise the peptide sequence Glu-Leu-Arg and a linker. In some embodiments, R can consist of only the peptide sequence Glu-Leu-Arg.

The linker is optional in some embodiments and can comprise, for example, any natural or non-natural amino acid. The amino acids can be represented by a formula: 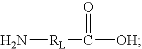
wherein R_{L} may be a substituted, unsubstituted, hetero-, straight-chained, branched, cyclic, saturated or unsaturated aliphatic radical; or a substituted, unsubstituted, or hetero- aromatic radical. In some embodiments, R_{L} can be substituted, unsubstituted, or hetero- forms of methyl, iso-propyl, sec-butyl, iso-butyl, benzyl, or a combination thereof.

In embodiments where R_{L} is substituted, examples of substitutents include, but are not limited to, hydroxyl, carboxyl, amino, imino groups and combinations thereof In embodiments where R_{L} is heteroaliphatic, examples of heteroatoms include, but are not limited to, sulfur, phosphorous, oxygen, nitrogen and combinations thereof In some embodiments, R_{L} can comprise substituted or unsubstituted poly(alkylene glycols), which include, but are not limited to, PEG, and PEG derivatives such as poly(ethylene oxide), poly(propylene glycol), poly(tetramethylene glycol), poly(ethylene oxide-co-propylene oxide), and copolymers and combinations thereof.

In some embodiments, R_{L} can be a substituted or unsubstituted alkylene comprising Cₙ carbons in the alkylene backbone, wherein n is an integer ranging from 1 to about 20; from about 2 to about 16; from about 3 to about 12; from about 4 to about 10; from about 3 to about 8, and any range therein. In these embodiments, R_{L} can be, for example, 11-amino-undecanoic acid.

In some embodiments, the linker comprises any combination of natural or non-natural amino acids, (Xaa), wherein the number of amino acids ranges from 1 to about 20; from about 2 to about 10; from about 3 to about 10, from about 1 to about 5, from about 1 to about 4, or any range therein. In some embodiments, the linker can comprise any combination of up to four natural or non-natural amino acids such as, for example, —(Gly)₄— (SEQ ID NO: 1640).

In some embodiments, there is no linker. In some embodiments, the mimetics are comprised of portions of the human CXC chemokine IP-10 that are connected directly to each other through amide bonds. In some embodiments, the mimetics are comprised of the human CXC chemokine IP-10 that are connected by disulfide bonds such as, the disulfide bonds that can form between Cys residues.

Any additional amino acids identified in square brackets in a header indicates that the peptide described by the header is at least an analog of a native fragment, and the position of the amino acid substitutions are noted by the standard abbreviation for the amino acid substituent with a superscript number indicating the residue positions at which the modification has occurred.

Methods of Administration

The terms “administer, “administration,” “administering,” and the like, can refer to a method of incorporating a compound into a subject to treat, prevent, or ameliorate the symptoms of, an abnormal condition in the subject. When a compound of the invention is provided in combination with one or more additional active agents, the administration can include administering the compound as a prodrug or codrug and can include sequential or concurrent administration with the other agent(s). One of skill will appreciate that any method of administration can be used in the practice of the present invention including, but not limited to, oral, injection, parenteral, dermal, and aerosol applications.

An “effective amount” of a compound of the invention includes a therapeutically effective amount or a prophylatically effective amount. A “therapeutically effective amount” refers to an amount effective, at dosages and for periods of time selectedby one of skill, to achieve the desired therapeutic result. The term “therapeutically effective amount” may also refer to that amount of active compound, prodrug or pharmaceutical agent that elicits a biological or medicinal response in a tissue, system, animal or human that is being sought by a researcher, veterinarian, medical doctor or other clinician in order to provide a therapeutic effect.

In some embodiments, a therapeutic effect relieves or prevents, to some extent, one or more of the symptoms of an abnormal condition. In some embodiments, a therapeutic effect can include, but is not limited to, one or more of the following: (a) an increase or decrease in the number of lymphocytic cells present at a specified location; (b) an increase or decrease in the ability of lymphocytic cells to migrate; (c) an increase or decrease in the response of lymphocytic cells to a stimulus; (d) an increase or decrease in the proliferation, growth, and/or differentiation of cells; (e) inhibition (i.e., slowing or stopping) or acceleration of cell death; (f) relieving, to some extent, one or more of the symptoms associated with an abnormal condition; (g) enhancing or inhibiting the function of the affected population of cells; (h) activating an enzyme activity present in cells associated with the abnormal condition; and (i) inhibiting an enzyme activity present in cells associated with the abnormal condition.

A “prophylactically effective amount” refers to an amount administered that is effective, at dosages and for periods of time determined readily by one of skill, at achieving a desired prophylactic result such as, for example, preventing or inhibiting a cytotoxic effect of a cytotoxic agent. Often, a prophylactic dose is used in organisms prior to, or at an early stage of, a disease, and in these embodiments, a prophylactically effective amount may be less than a therapeutically effective amount in some embodiments. The term “preventing” can refer, in some embodiments, to decreasing the probability that an organism contracts or develops an abnormal condition.

In some embodiments, the therapeutically or prophylactically effective amounts of chemokine analogs may range from about 0.1 nM-0.1 M, 0.1 nM-0.05 M, 0.05 nM-15 μM or 0.01 nM-10 μM, or any range therein. One of skill will appreciate that dosage values may vary with the severity of the condition to be alleviated. For any particular subject, specific dosage regimens may be adjusted over time according to the individual need and the professional judgement of the person administering or supervising the administration of the compositions. Accordingly, one of skill will also appreciate that the dosage ranges set forth herein are exemplary only and do not limit the dosage ranges that may be selected by medical practitioners.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

The term “dosage unit form” as used herein can refer to physically discrete units suited as unitary dosages for subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The terms “pharmaceutically acceptable carrier” or “excipient” can include any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions. Pharmaceutically acceptable carrier “may comprise pharmaceutically acceptable salts.”

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

Moreover, the chemokine analogs may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Additionally, suspensions may be prepared for injection. Suitable lipophilic solvents or vehicles include fatty oils such as sesame oil; or synthetic fatty acid esters, such as ethyl oleate or triglycerides; or liposomes. Suspensions to be used for injection may also contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical formulations for parenteral administration may include liposomes. Liposomes and emulsions are well known examples of delivery vehicles or carriers that are especially useful for hydrophobic drugs. Depending on biological stability of the therapeutic reagent, additional strategies for protein stabilization may be employed. Furthermore, one may administer the drug in a targeted drug delivery system, for example, in a liposome coated with target-specific antibody. The liposomes will bind to the target protein and be taken up selectively by the cell expressing the target protein.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof In accordance with an alternative aspect of the invention, a chemokine analog may be formulated with one or more additional compounds that enhance the solubility of the chemokine analog such as, for example, through pegylation.

If the compounds of the invention are to be administered by inhalation, they may be conveniently delivered in the form of an aerosol spray presentation from pressurized packs or a nebuliser, together with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin, for example, for use in an inhaler may be formulated containing a powder mix of the compound and a suitable powder base such as starch or lactose.

The following examples illustrate, but do not limit, the present invention.

### EXAMPLE 1

Peptides of the invention may be synthesized chemically using the Fmoc/tBu strategy on a continuous flow peptide synthesizer, as for example has been carried out using the following protocols:

Reagents (solvents, support, chemicals)

Main Solvent: N,N-Dimethylformamide (DMF): certified ACS spectroanalyzed from Fisher (D131-4) M.W=73.10. The DMF is treated with activated molecular sieves, type 4A (from BDH: B54005) for at least two weeks then tested with FDNB (2,4-Dinitrofluorobenzene from Eastman).

Procedure: Mix equal volumes of FDNB solution (1 mg/ml in 95% EtOH) and DMF; Let stand 30 minutes; read the absorbance at 381 nm over a FDNB blank (0.5 ml FDNB+0.5 ml 95% EtOH). If the absorbance ˜0.2, the DMF is suitable to be used for the synthesis.

Deblocking Agent: 20% Piperidine (from Aldrich Chemical company, catalog No: 10,409-4) in DMF containing 0.5 % Triton X100 v/v ( from Sigma, catalg No: T-9284).

Activating Agents: 2-(H-benzotriazol-lyl) 1,1,3,3-tetramethyluronium tetrafluoroborate (TBTU: M.W.=321.09. from Quantum Richilieu, catalog No: R0139)/Hydroxybenzotriazole (HOBt M.W.=135.1 from Quantum Richilieu, catalog No.: R0166-100) respectively, 0.52 M in DMF and 4-Methylmorpholine (NMM; M.W.=101.15, d=0.926 from Aldrich, catalog No.: M5,655-7): 0.9 M in DMF or in the case of sensitive amino acids to racemization like Cys, we use 2,4,6—Collidine, 99% (M.W.=121.18, d=0.917, from Aldrich, catalog No: 14,238-7): 0.78M in DMF/DCM, 1/1 v/v.

Support: TentaGel R RAM (90 μm), RinK-type Fmoc (from Peptides International, catalog No.: RTS-9995-PI): 0.21 mmol/g, 0.5 g for 0.1 mmol of peptide.

Fmoc-L-amino derivative, side-chains protected with: Boc; tBu; Trt groups: with 4 fold excess (from Peptides International, Bachem, Novabiochem, Chem-Impex Inc). Glu24 and Lys24 are Allyl-protected (from Millipore/Perseptive Biosystems).

Initial Amino Loading and Peptide Synthesis Procedure

The first amino acid Asn31 and the remaining residues are double coupled at room temp. or at 45° C. automatically with 4-fold excess in each coupling. The synthesis is interrupted after residue Leu19. The peptide-bound support is removed from the synthesizer column and placed in a react-vial containing a small magnetic bar for gentle stirring.

### Removal of the Allyl Groups

A solution of tetrakis(triphenylphosphine)Palladium(0) Pd(PPh3)4 (from Sigma-Aldrich, catalog No: 21,666-6); M.W.=1155.58×0.1 mmol peptide×3 fold=347 mg dissolved in 5% Acetic Acid; 2.5% NMM in CHCl3 to 0.14 M, under argon. The solution is added to the support-bound peptide previously removed from the coulmn in a reactvial containing a small mangnetic bar for gentle stirring. The mixture is flushed with argon, sealed and stirred at room temperature for 6 hours. The support-bound peptide is transferred to a filter funnel, washed with 30 ml of a solution made of 0.5% Sodium Diethyldithiocarbonate/in DMF, then DCM; DCM/DMF (1:1) and DMF. A positive Kaiser test indicates the deprotection of the amino side chaine of the Lys20.

### Lactam Formation:

Activating agent: 7-Azabenztriazol-1-yloxytris (pyrrolindino) phosphonium-hexafluorophosphate (PyAOP: M.W.=521.7 from PerSeptive Biosystems GmbH, catalog No: GEN076531), 1.4-fold: 0.105mmol×1.4×521.7=76.6 mg and NMM 1.5-fold: 0.105×1.4×1.5=0.23 mmol v=0.23/0.9 M NNM solution=263 μl).

The cychsation may be carried out in an amino acid vial at room temperature overnight (˜16 hours) with gentle agitation. The completion of cyclization may be indicated by a negative kaiser test. The support-bound peptide may be poured into the column, washed with DMF and the synthesis continues to completion, with a cyclic amide bridge thereby introduced into the peptide.

Final Product Removal From The Support: the support-bound peptide is removed from the synthesizer in to a medium filter funnel, washed with DCM to replace the non-volatile DMF and thoroughly dried under high vacuum for at least two hours, or preferably, overnight. Support: 0.5 g resin-peptide.

Cleavage Mixture (reagent K): 100 ml of a trifluoroacetic acid (TFA)/Phenol/Water/Thio-Anisol/EDT (82/5/5/5/2.5)(v/v) mixture is prepared. The support-bound peptide (0.5 g) is poured into 7.5 ml of reagent K with gentle agitation on a rocker, allowed to react for 4 hours at room temperature, filtered, and washed with neat TFA. The 7.5 ml of reagent K contains the following:

| | | |
|---|---|---|
| TFA | 6.15 | ml (Halocarbon) |
| Phenol | 0.375 | ml (Aldrich) |
| Water | 0.375 | ml (MillQ) |
| Thio-Anisol | 0.375 | ml (Aldrich) |
| EDT | 0.187 | ml (Aldrich) |
| Total | 7.5 | ml |

The cleavage may be performed at room temperature for 4 hours with gentle agitation on a rocker.

### Precipitation of the Peptide

The cleaved peptide solution is filtered through a filter funnel in a 50 ml round bottom flask. The support is rinsed twice with 4 ml TFA. The TFA solution is concentrated on a rotavap and added drop wise into a cold diethyl ether previously treated with activated neutral aluminum oxide to make it free of peroxide. Approximately 10-fold excess of ether are used. The beads are stored until the yield is determined and peptide characterized. The precipitate is collected at room temperature in screw capped 50 ml polypropylene vial by centrifugation at 2K rpm using a top bench centrifuge (4 minutes run time). The pellet is washed 3× with cold ether, centrifuged and dried with a flow of argon. The precipitate is dissolved in 20% acetonitrile, 0.1% TFA and lyophilized.

### Crude Product Characterization:

The product is characterized by analytical HPLC.

Experimental conditions: Column: Vydac 218TP54: C18 reversed-phase 5 μm, 4.6 mm ID×150mm L.

Eluants: 0.1% TFA/H₂O (solvant A); 0.1% TFA/acetonitrile (solvent B).

Elution Conditions: 20-50% B (40 min); 60-90% B (5 min); 90-20% B (5 min); 20% B (10 min). At 1.0 ml/min and A214 nm=0.5 absorbance unit full scale.

### Sample Preparation:

An aliquot of the product is weighed and dissolved in 20% acetonitrile 0.1% TFA at a concentration of 2 mg/ml. The solution is microfuged and 20 μl is applied onto the column. The main peak or the major peaks are collected, SpeedVac dried and molecular weight determined by mass spectrometry.

### EXAMPLE 2

The following sequences were prepared for testing their ability to bind to an IP-10 receptor and their efficacy in mediating intracellular calcium mobilization ([Ca²⁺]i) at a variety of concentrations:

Acetylated-IP-10-(1-16)-[linker]-IP-10-(66-78)
(SEQ ID NO:1641)
Ac-Val-Pro-Leu-Ser-Arg-Thr-Val-Arg-Cys-Thr-Cys-Ile-Ser-Ile-Ser¹⁵-
Asn-UDA-Leu⁶⁶-Lys-Ala-Val-Ser-Lys-Glu-Met-Ser-Lys-Arg-Ser-Pro
Acetylated-[Ala⁹,Phe¹¹]-IP-10-(1-16)-[linker]-IP-10-(66-78)
(SEQ ID NO:1642)
Ac-Val-Pro-Leu-Ser-Arg-Thr-Val-Arg-Ala⁹-Thr-Phe¹¹-Ile-Ser-Ile-Ser¹⁵-
Asn-UDA-Leu⁶⁶-Lys-Ala-Val-Ser-Lys-Glu-Met-Ser-Lys-Arg-Ser-Pro
[Pro7]-IP-10-(1-15)-[linker]-IP-10-(58-71)
(SEQ ID NO:1643)
Val-Pro-Leu-Ser-Arg-Thr-Pro⁷-Arg-Cys-Thr-Cys-Ile-Ser-Ile-Ser¹⁵-
UDA-Glu⁵⁸-Ser-Lys-Ala-Ile-Lys-Asn-Leu-Leu-Lys-Ala-Val-Ser-Lys
[Ser⁹,Ser¹¹,Glu⁶³]-IP-10-(1-15)-[linker]-IP-10-(58-71)
(SEQ ID NO:1644)
Val-Pro-Leu-Ser-Arg-Thr-Val-Arg-Ser⁹-Thr-Ser¹¹-Ile-Ser-Ile-Ser¹⁵-
UDA-Glu⁵⁸-Ser-Lys-Ala-Ile-Glu⁶³-Asn-Leu-Leu-Lys-Ala-Val-Ser-Lys
[Ser⁹,Ser¹¹,Glu⁶⁷]-IP-10-(1-15)-[linker]-IP-10-(58-71)
(SEQ ID NO:1645)
Val-Pro-Leu-Ser-Arg-Tbr-Val-Arg-Ser⁹-Thr-Ser¹¹-Ile-Ser-Ile-Ser¹⁵-
UDA-Glu⁵⁸-Ser-Lys-Ala-Ile-Lys-Asn-Leu-Leu-Glu⁶⁷-Ala-Val-Ser-Lys

wherein, UDA is 11-amino undecanoic acid.

Binding and Calcium Mobilization: Suspensions of CXCR-3/300-19 cells were used to assess binding and intracellular calcium mobilization induced by IP-10 analogs. These are mouse pre-B lymphocytes transfected with the CXCR3 receptor, (Moser, et al). The cells were washed in RPMI media and resuspended in RPMI media supplemented with 10% FCS, then plated at 1.2×10⁵ cells per well of 96-well black wall/clear bottom plates coated with poly-D-lysine (Becton Dickinson) and loaded with 100 μL fluorescent calcium indicator FLIPR Calcium 3 assay kit component A (Molecular Probes) for lhr at 37° C. The cells on the plates were then spun at 1000 rpm for 15 minutes at room temperature.

Each of the sequences successfully bound to the cellular receptors. The intracellular calcium mobilization in response to 25 μL (0-100000 nM final concentrations) of the appropriate and various concentrations of analogue was measured at 37° C. by monitoring fluorescence as a function of time in all the wells using the

Flexstation Fluorometric Imaging Plate Reader (Molecular Devices). All analogues were run simultaneously with rhIP-10 (R&D Systems) as the standard. Table 1 provides the dosage effect of the binding of each of the IP-10 analogs on the calcium mobilization activity of the cells.

TABLE 1

| Peptide Analogs | Dosage (μM) | | | | |
|---|---|---|---|---|---|
| (SEQ ID NO:) | 1.2 | 3.7 | 11.1 | 33.3 | 100 |
| 1641 | 12.6 | 12.7 | 14.1 | 20 | 52.9 |
| 1642 | 15.2 | 13.8 | 15.4 | 20 | 3.1 |
| 1643 | 16 | 14 | 14.4 | 18.6 | 55.1 |
| 1644 | 18.8 | 18.5 | 17.2 | 18.9 | 46.6 |
| 1645 | 17.4 | 15.4 | 12.5 | 13.3 | 3.4 |

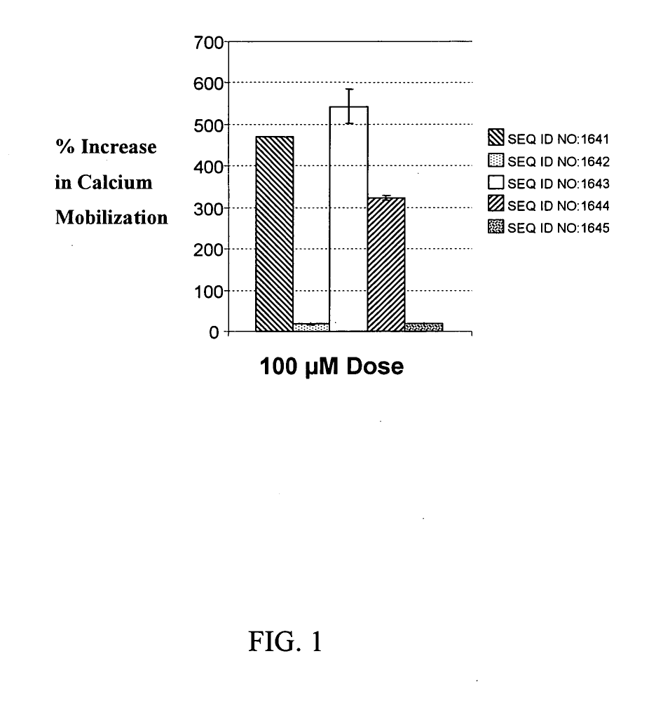

FIG. 1 illustrates the induction of [Ca²⁺]i mobilization by select IP-10 analogs at a concentration of 100 μM according to some embodiments of the present invention. The results are representative of three independent experiments. SEQ ID NOs: 1641-1645 all bound to the receptor and affected calcium mobilization. SEQ ID NOs: 1641, 1643, and 1644, however, increased calcium mobilization by 300 to over 500%. The results are compared to a recombinant human IP-10 chemokine, as described above.

The acetylated-IP-10-(1-16)-[linker]-IP-10-(66-78) analog represented by SEQ ID NO: 1641 increased intracellular calcium mobilization by nearly 500%, but an [Ala⁹,Phe¹¹] amino acid substitution in the same type of analog decreased the effect on calcium mobilization dramatically as shown by the effects of SEQ ID NO: 1642.

In an IP-10-(1-15)-[linker]-IP-10-(58-71) analog, a [Pro⁷] amino acid substitution in SEQ ID NO: 1643 resulted in an increase in intracellular calcium mobilization when compared to the results of SEQ ID NO: 1641. A [Ser⁹,Ser¹¹,Glu⁶³] amino acid substitution of the same type of analog still provide a very substantial increase in intracellular calcium mobilization of over 300% using SEQ ID NO: 1644. Interestingly, however, a [Glu⁶⁷] amino acid substitution decreased the effect on calcium mobilization dramatically as shown by the effect of SEQ ID NO: 1645.

The results provided by this example show that IP-10 analogs having a total of about 30 amino acids and conserving N-terminal residues 1-15 and C-terminal residues 66-71 of the IP-10 chemokine are effective at binding and can increase the cellular activity induced by the binding to different degrees, depending on the dosage of the analog administered and the presence of amino acid substitutions. In particular, the results suggest that the Cys⁹ and Cys¹¹ residues can be substituted with Ser⁹ and Ser¹¹ in the conserved N-terminal 1-15 region with little effect, and Lys⁶³ can be substituted by Glu⁶³ with little effect, but substantial differences in results occur where Lys⁶⁷ is substituted with Glu⁶⁷, which is in the range of the conserved C-terminal region of 66-71.

Accordingly, an IP-10 analog that is supported by these results would range from about 21 to about 34 amino acids in length and comprise:
an N-terminal region comprising and conserving the IP-10 chemokine residues 1-15
R-Val-Pro-Leu-Ser-Arg-Thr-Val-Arg-Cys-Thr-Cys-Ile-Ser-Ile-Ser¹⁵-
(OH)NH₂
(SEQ ID NO: 1646), and conservatively modified variants thereof,
a C-terminal region comprising and conserving the IP-10 chemokine residues 66-71, and conservatively modified variants thereof,
Leu⁶⁶-Lys-Ala-Val-Ser-Lys⁷¹-(OH)NH₂
(SEQ ID NO: 1647), and conservatively modified variants thereof,
and an optional linker having up to 4 amino acids, wherein the linker is preferably 11-aminoundecanoic acid.

### EXAMPLE 3

An IP-10 can be modified with a polysaccharide such as heparin. The glycosaminoglycan can be connected to an amine functional group as an aldehyde-terminated heparin. An example of an aldehyde-terminated heparin is represented by the following formula: 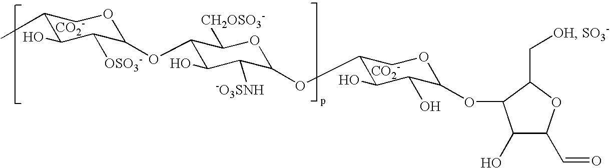
wherein p is an integer not equal to 0.

The aldehyde-terminated heparin can be combined with the amine functional group in a DMF/water solvent and subsequently reduced with NaCNBH₃ to produce the following structure:

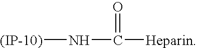

### EXAMPLE 4

An IP-10 mimetic can be modified with a polyalkylene glycol using a variety of techniques. There are a variety of available PEG sizes and derivatives that are commercially designed for specific applications such as, for example, attachment to a variety of different chemical functionalities including, but not limited to, amines, thiols, hydroxyls, sulfhydryls, and carboxyls.

In one example, an amine group of an IP-10 can be combined with a carboxyl-terminated PEG (Nektar Corp.) in the presence of, for example, EDC or DCC to form the following structure: 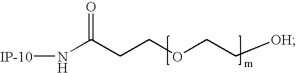
wherein m is an integer not equal to 0.

In another example, either a succinimidyl derivative of mPEG (Nektar Corp.) or an isocyanate-terminated mPEG (Nektar Corp.) can be combined with an IP-10 mimetic under conditions known to those of skill in the art. In another example, the carboxyl group of an IP-10 mimetic can be activated with, for example, EDC or DCC and combined with an amino-terminated mPEG (Nektar Corp.) In another example, an amine group of an IP-10 mimetic can be combined with a methacrylate-terminated mPEG (Nektar Corp.) in the presence of an initiator capable of undergoing thermal or photolytic free radical decomposition. Examples of suitable initiators include benzyl-N,N-diethyldithiocarbamate or p-xylene-N,N-diethyldithiocarbamate.

The invention is not limited in its application to the details of structures and the arrangements of components set forth herein. It should be understood that although the present invention has been disclosed using select embodiments and optional features, modifications and variations of the inventions can be readily made by those skilled in the art, and that such modifications and variations are considered to be within the scope of the inventions.

The inventions have been described broadly and generically herein, and Applicant includes an inherent proviso, or negative limitation, that will allow removing any species subject matter from genus teachings, regardless or whether or not the material to be removed was specifically recited or stated as being removable. Some changes can be made in the form and detail of the teachings by one of skill without departing from the spirit and the scope of the invention. All patents and publications described herein are hereby incorporated by reference to the same extent as if each individual patent or publication was specifically and individually indicated to be incorporated by reference herein in its entirety.

We claim:

1. A composition comprising an IP-10 chemokine analog having a length ranging from about 21 to about 34 amino acids and comprising:
a first conserved sequence consisting of the IP-10 chemokine residues 1-15,
Val¹-Pro-Leu-Ser-Arg-Thr-Val-Arg-Cys-Thr-Cys-Ile-Ser-Ile-Ser¹⁵
(SEQ ID NO: 1646), and conservatively modified variants thereof;
a second conserved sequence consisting of the IP-10 chemokine residues 66-71,
Leu⁶⁶-Lys-Ala-Val-Ser-Lys⁷¹
(SEQ ID NO: 1647), and conservatively modified variants thereof; and,
an optional linker having up to 4 amino acids; wherein,
the N-terminus of the IP-10 analog consists of a hydrogen or is modified using an N-terminal modifier comprising a component selected from a group consisting of a poly(ethylene glycol) or derivative thereof, a glycosarninoglycan, a diagnostic label, a radioactive group, an acyl group, an acetyl group, a peptide, and a modifier capable of reducing the ability of the IP-10 analog to act as a substrate for aminopeptidases;
and the linker is selected from a group consisting of (a) up to four natural amino acids, and (b) any non-natural amino acid having the following structure: 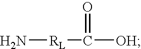
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

2. The composition of claim 1 having a length of about 26 to 32 amino acid residues and comprising:
an N-terminal region consisting essentially of SEQ ID NO: 1646 and conservatively modified variants thereof in residues 1-15, and
a C-terminal region comprising SEQ ID NO: 1647 and conservatively modified variants thereof

3. The composition of claim 1, wherein the linker is 11-aminoundecanoic acid.

4. The analog of claim 1 comprising an amino acid sequence selected from a group consisting of SEQ ID NO: 1641, SEQ ID NO: 1642, SEQ ID NO: 1643, SEQ ID NO: 1644, and SEQ ID NO: 1645.

5. The composition of claim 1 comprising an amino acid sequence selected from a group consisting of SEQ ID NOs:296-349 and 404-457, variants b134-b187 and b242-b295, inclusive; wherein, Xaa₁, Xaa₂, Xaa₃, and Xaa₄ are each independently selected from a group consisting of any natural amino acid or any non-natural amino acid having the following structure: 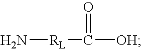
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

6. The composition of claim 5, wherein the linker is 11-aminoundecanoic acid.

7. The composition of claim 1 comprising an amino acid sequence selected from a group consisting of SEQ ID NOs:494-548 and 675-728, variants b332-b385 and b512-b566, inclusive; wherein, Xaa₁, Xaa₂, Xaa₃, and Xaa₄ are each independently selected from a group consisting of any natural amino acid or any non-natural amino acid having the following structure: 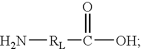
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

8. The composition of claim 5, wherein the linker is 11-aminoundecanoic acid.

9. A method of increasing the IP-10-mediated activity of a cell having a receptor capable of binding to an IP-10 analog, comprising:
binding the receptor to an IP-10 analog having a length ranging from about 21 to about 34 amino acids and comprising:
a first conserved sequence consisting of the IP-10 chemokine residues 1-15,
Val¹-Pro-Leu-Ser-Arg-Thr-Val-Arg-Cys-Thr-Cys-Ile-Ser-Ile-Ser¹⁵
(SEQ ID NO: 1646), and conservatively modified variants thereof;
a second conserved sequence consisting of the IP-10 chemokine residues 66-71,
Leu⁶⁶-Lys-Ala-Val-Ser-Lys⁷¹
(SEQ ID NO: 1647), and conservatively modified variants thereof; and,
an optional linker having up to 4 amino acids; wherein,
the N-terminus of the IP-10 analog consists of a hydrogen or is modified using an N-terminal modifier comprising a component selected from a group consisting of a poly(ethylene glycol) or derivative thereof, a glycosaminoglycan, a diagnostic label, a radioactive group, an acyl group, an acetyl group, a peptide, and a modifier capable of reducing the ability of the IP-10 analog to act as a substrate for aminopeptidases;
and the linker is selected from a group consisting of (a) up to four natural amino acids, and (b) any non-natural amino acid having the following structure: 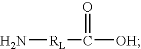
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

10. The method of claim 9, wherein the IP-10 analog has a length of about 26 to 32 amino acid residues and comprises:
an N-terminal region consisting essentially of SEQ ID NO: 1646 and conservatively modified variants thereof in residues 1-15, and
a C-terminal region comprising SEQ ID NO: 1647 and conservatively modified variants thereof.

11. The method of claim 9, wherein the linker is 11-aminoundecanoic acid.

12. The method of claim 9, wherein the IP-10 analog comprises an amino acid sequence selected from a group consisting of SEQ ID NO: 1641, SEQ ID NO: 1642, SEQ ID NO: 1643, SEQ ID NO: 1644, and SEQ ID NO: 1645.

13. The method of claim 9 comprising an amino acid sequence selected from a group consisting of SEQ ID NOs:296-349 and 404-457, variants b134-b187 and b242-b295, inclusive; wherein, Xaa₁, Xaa₂, Xaa₃, and Xaa₄ are each independently selected from a group consisting of any natural amino acid or any non-natural amino acid having the following structure: 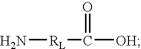
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

14. The method of claim 13, wherein the linker is 11-aminoundecanoic acid.

15. The method of claim 9 comprising an amino acid sequence selected from a group consisting of SEQ ID NOs:494-548 and 675-728, variants b332-b385 and b512-b566, inclusive; wherein, Xaa₁, Xaa₂, Xaa₃, and Xaa₄ are each independently selected from a group consisting of any natural amino acid or any non-natural amino acid having the following structure: 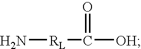
wherein, R_{L} is selected from a group consisting of saturated and unsaturated aliphatics and heteroaliphatics consisting of 20 or fewer carbon atoms that are optionally substituted with a hydroxyl, carboxyl, amino, amido, or imino group; or an aromatic group having from 5 to 7 members in the ring; and —(CH₂)ₙ—, wherein n is an integer ranging from 1 to 20.

16. The method of claim 15, wherein the linker is 11-aminoundecanoic acid.

17. An antibody produced using the IP-10 analog of claim 1 as an antigen.

18. The antibody of claim 17, wherein the antibody is monoclonal.

19. The antibody of claim 17, wherein the antibody is humanized.

20. The antibody of claim 17, wherein the antigen is the IP-10 analog of claim 4.
